(19) United States
(12) Patent Application Publication
(10) Pub. No.: US 2005/0009877 A1
(43) Pub. Date: Jan. 13, 2005

(54) Methods of identifying HCV NS5B polymerase inhibitors and their uses

(76) Inventor: Henry Lu, Foster City, CA (US)

Correspondence Address: DORSEY & WHITNEY LLP;INTELLECTUAL PROPERTY DEPARTMENT, 4 EMBARCADERO CENTER, SUITE 3400, SAN FRANCISCO, CA, 94111, US

(21) Appl. No.: 10/847,822

(22) Filed: May 17, 2004

Related U.S. Application Data
(60) Provisional application No. 60/471,444, filed on May 15, 2003.

(51) Int. Cl. C12Q 1/70; A61K 31/4439; A61K 31/433; A61K 31/4245; A61K 31/4196

(52) U.S. Cl. 514/341; 514/362; 514/364; 514/383; 435/5

(57) ABSTRACT

The present invention relates to a variety of screening methods, utilizing both biochemical and cellular assays as well as in silicon assays, for use in the discovery of agents active in the treating or preventing Hepatitis C virus (HCV) infections. The invention also relates to methods of inhibiting an HCV NS5B polymerase and to the treatment and/or prevention of HCV infections with compounds having specified binding properties.

### 1. CROSS REFERENCE TO RELATED APPLICATIONS

This application claims benefit under 35 U.S.C. § 119(e) to provisional application Ser. No. 60/471,444 filed May 15, 2003, the disclosure of which is incorporated herein by reference in its entirety.

### 2. FIELD OF INVENTION

The present invention relates to a variety of biochemical, cellular and in silico screening methods and assays for use in the discovery of agents active in the treatment and/or prevention of Hepatitis C virus (HCV) infections, as well as to molecules having specified properties and their use to inhibit hepatitis C virus replication and/or proliferation and/or treat or prevent HCV infections.

### 3. BACKGROUND OF THE INVENTION

Hepatitis C virus (HCV) infection is a global human health problem with approximately 150,000 new reported cases each year in the United States alone. HCV is a single stranded RNA virus, which is the etiological agent identified in most cases of non-A, non-B post-transfusion and post-transplant hepatitis and is a common cause of acute sporadic hepatitis (Choo et al., Science 244:359, 1989; Kuo et al., Science 244:362, 1989; and Alter et al., in Current Perspective in Hepatology, p. 83, 1989). It is estimated that more than 50% of patients infected with HCV become chronically infected and 20% of those develop cirrhosis of the liver within 20 years (Davis et al., New Engl. J. Med. 321:1501, 1989; Alter et al., in Current Perspective in Hepatology, p. 83, 1989; Alter et al., New Engl. J. Med. 327:1899, 1992; and Dienstag Gastroenterology 85:430, 1983). Moreover, the only therapy available for treatment of HCV infection is interferon-α (INTRON® A, PEG-INTRON® A, Schering-Plough; ROFERON-A®, PEGASyse®, Roche). Most patients are unresponsive, however, and among the responders, there is a high recurrence rate within 6-12 months after cessation of treatment (Liang et al., J. Med. Virol. 40:69, 1993). Ribavirin, a guanosine analog with broad spectrum activity against many RNA and DNA viruses, has been shown in clinical trials to be effective against chronic HCV infection when used in combination with interferon-α (see, e.g., Poynard et al., Lancet 352:1426-1432, 1998; Reichard et al., Lancet 351:83-87, 1998), and this combination therapy has been recently approved (REBETRON, Schering-Plough; see also Fried et al., 2002, N. Engl. J. Med. 347:975-982). However, the response rate is still at or below 50%.

The crystal structure of the RNA dependent RNA polymerase (RdRp), also referred to as NS5B, has been published; see U.S. Pat. No. 6,434,489; Ago et al., 1999, Structure 7:1417 (coordinates deposited in the Protein Data Bank with accession code 1quv); Lesburg et al., 1999, Nature Structural Biology 6:937 (coordinates deposited in the Protein Data Bank with accession code 1C2P); and Bressanelli et al., 1999, Proc. Natl. Acad. Sci. 96:13034-13039 (coordinates deposited with the Protein Data Bank with assession code 1 CSJ), all of which are expressly incorporated herein by reference. The HCV RdRp protein is divided into three domains (the finger domain, the palm domain and the thumb domain), on the basis of its resemblance to a wide variety of other known polymerases including Taq DNApol1, and others described in Ago et al., supra.

In addition, there are a number of known genotypes of different HCV isolates, as is more fully described below.

### 4. SUMMARY OF THE INVENTION

Recently, several novel classes of potent HCV inhibitors have been identified, which are more fully described in the detailed description section and the various copending applications and publication listed below. These classes share certain structural similarities and are generally characterized by three main features: (i) a substituted 6-membered aromatic “A” ring; (ii) a substituted or unsubstituted 5-membered saturated, unsaturated or aromatic “B” ring; and (iii) a substituted 6-membered aromatic “C” ring. These rings are generally connected to one another as follows:

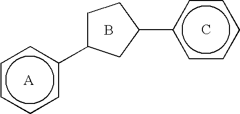

Note that the depiction of the “A”, “B” and “C” rings in this format is merely for schematic purposes only and is not meant to exclude the use of heteroatoms within any of these rings. Indeed, in many embodiments one or both of the “A” and “C” rings includes a nitrogen heteroatom and the “B” ring includes from one to four of the same or different heteroatoms selected from N (or NH), O and S.

In many of these compounds, the “C” ring is substituted at the meta position with a gem-dihaloacetamide group of the formula —NR¹¹—C(O)CHXX, where R¹¹ is hydrogen or alkyl and each X is the same or different halo group and may also include one or more of the same or different substituent groups at the other ring positions. In a specific embodiment, R¹¹ is hydrogen and each X is the same halo group, preferably chloro.

The “A” ring includes at least one substituent positioned ortho (2- or 6-position) and may optionally include one or more of the same or different substituents positioned at the other ring positions. In some embodiments, the “A” ring bears that same or different substituents at the 2- and 6-positions and is unsubstituted at the 3-, 4- and 5-positions.

Exemplary embodiments of the HCV inhibitory compounds include compounds in which both of the “A” and “C” rings are substituted phenyl groups, compounds in which one or both of the “A” and “C” rings are pyridyl groups, for example pyrid-2-yl groups, and compounds in which the “B” ring is an aromatic ring comprising one, two or three heteroatoms or heteroatomic groups selected from N, NH, O and S, including, for example, isoxazoles, pyrazoles, triazoles and oxadiazoles. These various classes of compounds, including various prodrugs, solvates, oxides and salts thereof, as well as specific species of these compounds and methods for their synthesis, are described in the following copending applications: international application No. PCT/US02/35131 filed May 15, 2003 (WO 03/040112); U.S. application Ser. No. 10/286,017 filed Sep. 4, 2003 (publication No. U.S. 2003/0165561); U.S. application Ser. No. 60/467,650 filed May 2, 2003; U.S. application Ser. No.______ filed Apr. 30, 2004 (identified as attorney docket no. 28569/US/US/2); international application No.______ filed Apr. 30, 2004 (identified as attorney docket no. 28569/US/PCT/2); U.S. application Ser. No. 60/467,811 filed May 2, 2003; U.S. application Ser. No. 10/838,133 filed May 3, 2004; U.S. application Ser. No. 10/440,349 filed May 15, 2003; U.S. application Ser. No. 10/646,348 filed Aug. 22, 2003; and international application No. PCT/US03/026478 filed Aug. 22, 2003 (WO 2004/018463). The disclosures of these applications are incorporated herein in their entireties.

It has now been discovered, as confirmed in biochemical assays with representative compounds, that these novel HCV inhibitors bind the NS5B polymerase of HCV. In addition, it has been discovered that these compounds associate with specified amino acid residues in a particular pocket of the NS5B polymerase. Specifically, NSSB mutations identified in replicons resistant to the exemplary species Compounds A, B and C (See FIG. 10 and FIG. 2) reveal that these classes of compounds likely contact, associate with, and/or interact with one or more amino acid residues at the following positions of the NSSB polymerase: 142, 148, 213, 316, 444, 445, 447, 451, 452 and/or 465 (using the numbering system of Bressanelli et al., 1999, Proc. Natl. Acad. Sci. USA 96:13034-13039).

Many of these residues are highly conserved. For example, out of 156 clinical NS5B polymerase isolates sequenced, 88 have an Asn at residue position 110 (Asn¹¹⁰), 51 have a Ser at this position (Ser¹¹⁰; “wild-type”), 14 have a Cys at this position (Cys¹¹⁰) and 3 have a Gly at this position (Gly¹¹⁰); 142 have an Asn at position 142 (Asn¹¹⁰; “wild-type”) and 26 have a Ser at this position (Ser¹⁴²); 155 have a Tyr at position 452 (Tyr⁴⁵²; “wild-type”) and one has a His at this position (His⁴⁵²); and all 156 have an Arg at position 465 (Arg⁴⁶⁵; “wild-type”). Moreover, when superimposed on a crystal structure of an NS5B polymerase, these residues map to a pocket which is defined in part by certain structural elements that reside in the “thumb” subdomain, as will be described in more detail, below (see FIG. 1). Although it has been speculated that this pocket, referred to herein as the “Rigel pocket” is involved in a number of essential biochemical functions, including the oligomerization of the NS5B polymerase, the interaction of the NS5B polymerase with other HCV proteins and the binding of RNA (the latter based on structural analogy to the HIV reverse transcriptase protein), this pocket and its associated residues has never before been confirmed or identified as a target for HCV inhibitory compounds.

Quite significantly, this pocket and its specified residues reside in a different region of the NS5B polymerase than that bound by other known inhibitors of the NS5B polymerase, such as the two non-nucleoside inhibitors (2S)-2-[(2,4-dichloro-benzoyl)-(3-trifluromethyl-benzyl)-amino]-3-phenyl-propionic acid and (2S)-2-[(5-benzofuran-2-yl-thiopen-2-yl-methyl)-(2,4-dichloro-benzoyl)-amino]-3-phenyl-propionic acid. As reported in the literature, these two inhibitors bind a common pocket located exclusively in the “thumb” subdomain of the NS5B polymerase (see, Wang et al., 2003, J. Biol. Chem. 278:9489-9495).

The identification of this new Rigel pocket provides a powerful mechanism by which the NS5B can be inhibited and HCV infections may be treated and/or prevented. It also provides a powerful new tool for the identification and/or design of new compounds useful to inhibit HCV replication, and in particular compounds useful to treat and/or prevent HCV infections. The present disclosure provides myriad different methods that capitalize on this important discovery.

In one aspect, the present disclosure provides a method of inhibiting an HCV NS5B polymerase utilizing compounds which bind the Rigel pocket of NS5B polymerase. The method generally comprises contacting an NS5B polymerase with an amount of a Rigel pocket binding compound (sometimes referred to herein as a “pocket binding inhibitor” or “PBI”) effective to inhibit an activity of the NS5B polymerase. The pocket binding compound may bind any region of the Rigel pocket, and may optionally and preferably contact, interact with and/or associate with one or more of the NS5B amino acid residues at positions 142, 148, 213, 316, 444, 445, 447, 451, 452 and 465, with contacts with at least one of residues 452 and 465 being especially preferred. The activity inhibited may be any known or later-discovered activity associated with the Rigel pocket. The methods may be used in a variety of contexts, including in vitro, in vivo and ex vivo contexts to inhibit the NS5B polymerase. In some embodiments, the methods may be used in in vitro or in vivo contexts to inhibit HCV replication and/or proliferation. In another embodiment, the methods may be used in in vivo contexts as a therapeutic approach towards the treatment and/or prevention of HCV infections.

In another aspect, the present disclosure provides a method of inhibiting HCV replication and/or proliferation. The method generally comprises contacting a hepatitis C virion with an amount of a Rigel pocket binding compound (sometimes referred to herein as a “pocket binding inhibitor” or “PBI”) effective to inhibit the replication and/or proliferation of the hepatitis C virion. The method may be used in a variety of contexts, including in vitro, in vivo and ex vivo contexts to inhibit HCV replication and/or proliferation. In some embodiments, the methods may be used as in in vivo contexts as a therapeutic approach towards the treatment and/or prevention of HCV infections.

In yet another aspect, the present disclosure provides a method of treating or preventing an HCV infection. The method generally comprises administering to a subject in need thereof an amount of a Rigel pocket binding compound (sometimes referred to herein as a “pocket binding inhibitor” or “PBI”) effective to treat or prevent the HCV infection. The method may be practiced therapeutically in subjects suffering from an HCV infection, or prophylactically in subjects thought to be at risk of developing an HCV infection, whether actually exposed to HCV or not. For example, the therapy may be administered to hospital workers or patients accidentally stuck with needles, regardless of whether the needle is contaminated with HCV.

In still another aspect, the present disclosure provides methods of screening for and/or identifying additional compounds that bind, associate with or interact with the Rigel pocket of an HCV NS5B polymerase. In general, the methods comprise contacting an NS5B polymerase with a candidate agent and determining whether the candidate agent binds, associates with and/or interacts with the Rigel pocket of the NS5B polymerase. In some embodiments, it is determined whether the candidate agent binds, associates with and/or interacts with one or more of the NS5B amino acid residues at the following positions: 142, 148, 213, 316, 444, 445, 447, 451, 452 and 465.

The contacting can be carried out in vitro using real candidate agents and NS5B polymerase, or in silico using structures or atomic structure coordinates of the candidate agent and NS5B polymerase. When carried out in vitro, a variety of methods may be used, including heterogeneous assays as well as competitive binding assays with known PBIs. In optional embodiments, either or both of the NS5B polymerase and candidate agent may be attached to a solid support, or either or both of the NS5B polymerase and candidate agent may be labeled for ease of detection. Alternatively, the assay may be carried out using spectroscopic methods, such as NMR spectroscopy.

When carried out in silico, any of the art-known computer programs designed for in silico screening of compounds may be employed. The methods may be carried out with the atomic structure coordinates of the entire NS5B polymerase, or alternatively with the coordinates of only specified residues, such as the residues that define the pocket or the pocket residues involving specified contacts.

Additional assays are provided which test the pocket region binding candidate agents as modulators of any of the bioactivities of the NS5B polymerase.

In yet another aspect, the present disclosure provides methods of designing PBI compounds. The methods generally employ well known in silico techniques utilizing, for example, fragment assembly, but may also employ other well-known techniques, such as NMR. The PBI compounds may be designed to contact, associate with and/or interact with one or more of the specified NS5B polymerase residues described above. Like the in silico screening methods, the in silico design methods may employ atomic structure coordinates of all or a portion of the NS5B polymerase.

### 5. BRIEF DESCRIPTION OF THE FIGURES

FIG. 1 depicts the general schematic structure of HCV NS5B and the location of some important pocket region residues.

FIG. 2 depicts some of the mutations of NS5B identified in the viral drug resistance screens described in the Examples.

FIG. 3 depicts the direct binding of Compound A (illustrated in FIG. 10) with HCV NS5B as determined by a standard Biacore assay.

FIG. 4 depicts the fluorescence quenching of the inherent fluorescence emission of NS5B upon binding of Compound A (illustrated in FIG. 10).

FIG. 5 shows the rate of emergence of drug resistant clones.

FIG. 6A shows that the replication of Compound A resistant clone A-1 is less sensitive to inhibition by exemplary PBI compounds than the present non-resistant replicon.

FIG. 6B shows that the replication of Compound C-resistant-clone C-3A is less sensitive to inhibition by exemplary PBI compounds than the present non-resistant replicon.

FIG. 7 shows the alignment of NS5B sequences from different HCV genotypes.

FIGS. 8A and 8B show that Compound A inhibits two biochemical properties of NS5B, including the de novo synthesis of RNA and the RNA chain elongation assay.

FIGS. 9A, 9B and 9C depict the effects of different reducing agents on the activity of NS5B.

FIGS. 10A, 10B, 10C and 10D depict various PBIs, Compounds A, B, C and D, respectively.

FIGS. 11A-11K recite the Genbank accession numbers for a wide variety of complete HCV genomes, from which the NS5B is easily identified via homology studies, and all of which are incorporated by reference, particularly to the extent that there are sequence differences in the NS5B polymerase sequences between these genotypes.

FIG. 12 corresponds to FIG. 2 of Brassenelli et al., 1999, Proc. natl. Acad. Sci. USA 96:13034-13039 and depicts sequence and structural alignments of HCV NS5B polymerase (line labeled HCV1) with polymerases and reverse transcriptases from other sources. The various beta strands (numbered) and alpha helices (lettered) are indicated by solid symbols above the sequences. The other features depicted in the figure are described in Brassenelli et al.,supra.

### 6. DETAILED DESCRIPTION OF THE PREFERRED EMBODIMENTS

6.1 Definitions

As used herein, the following terms are intended to have the following meanings:

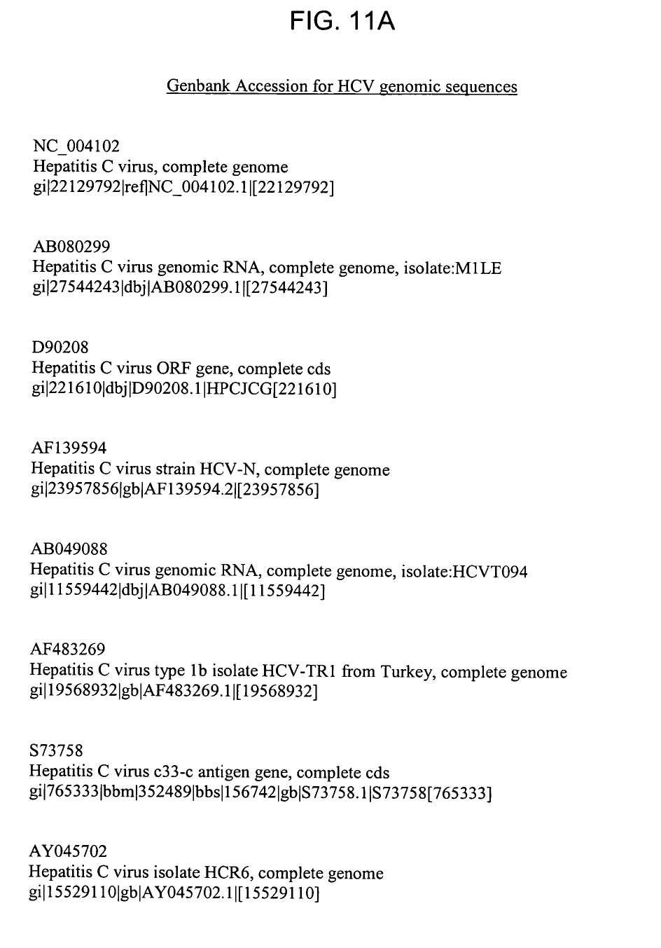

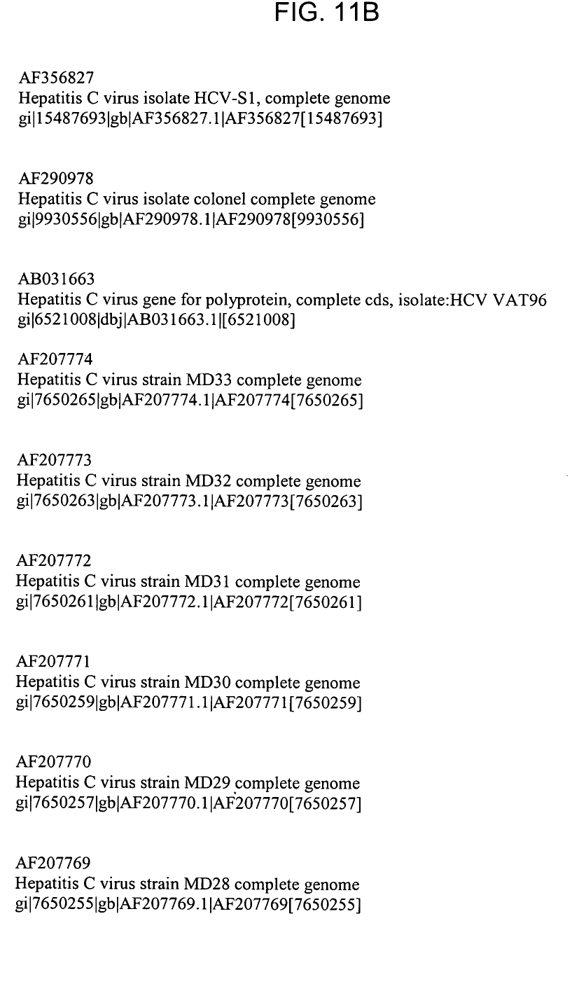

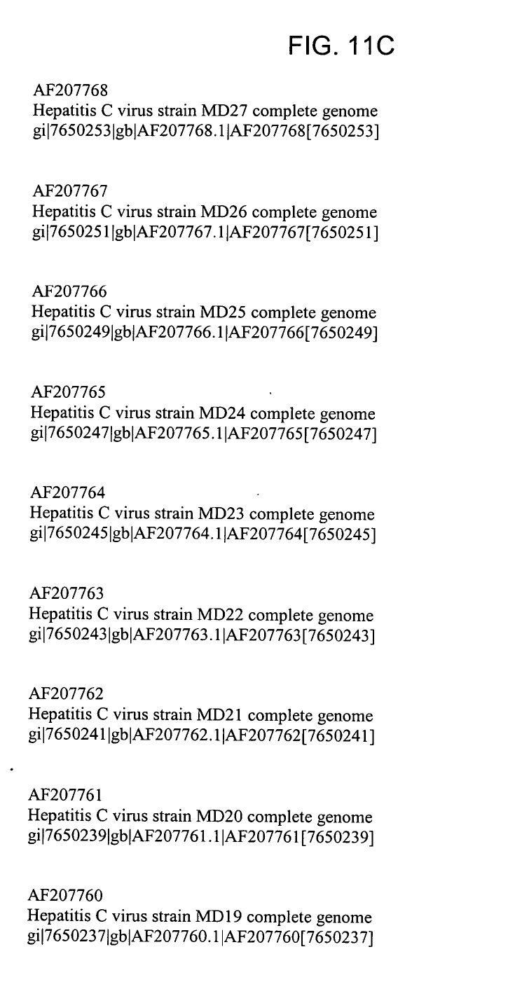

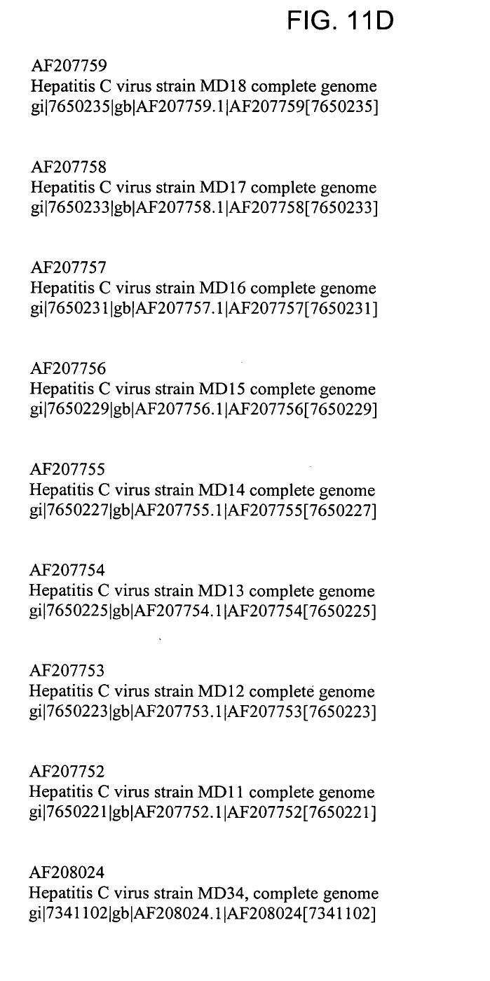

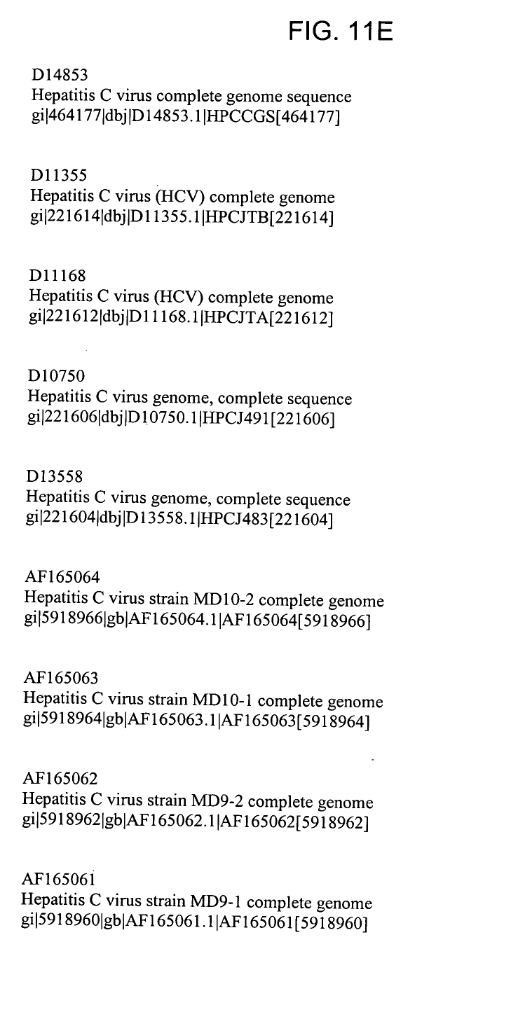

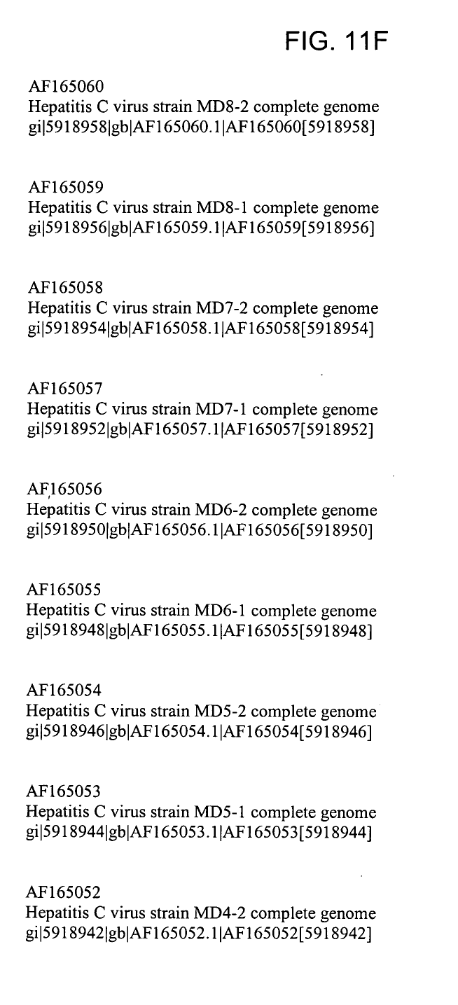

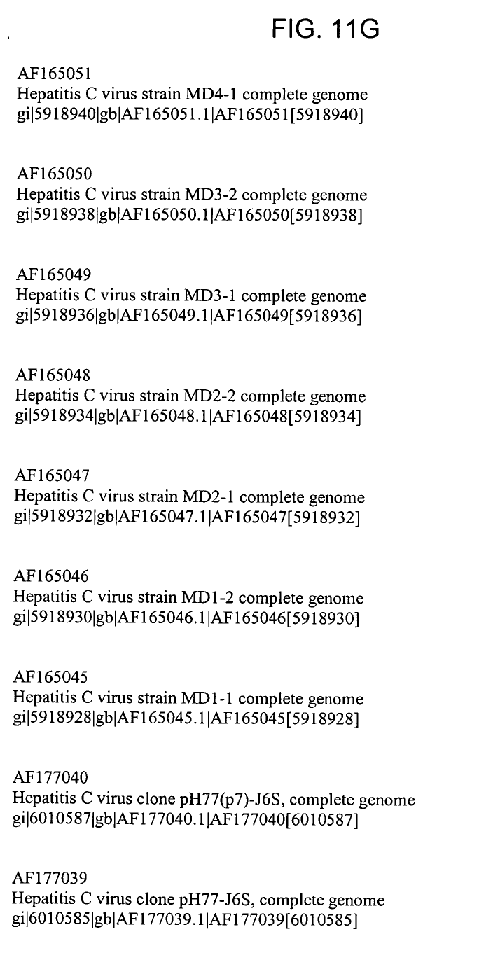

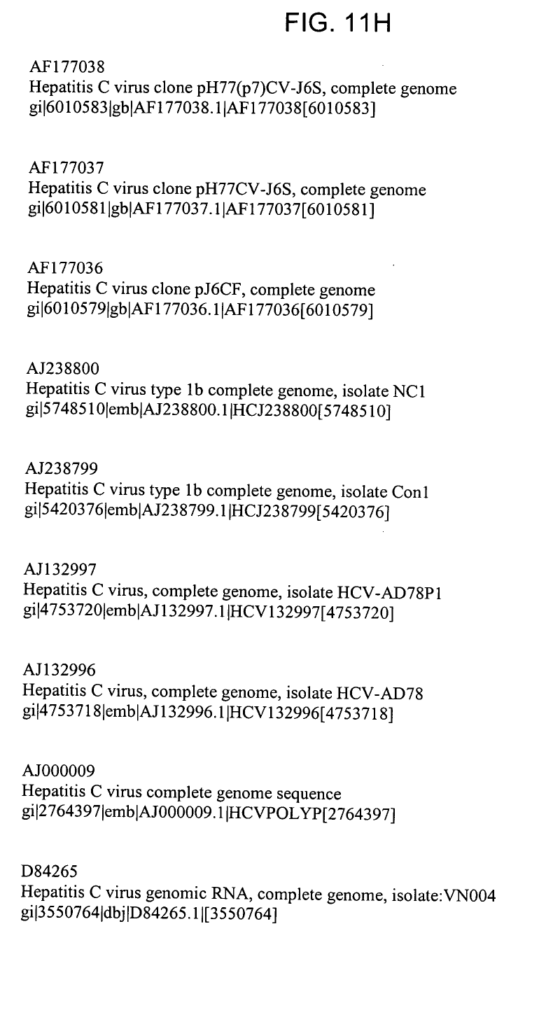

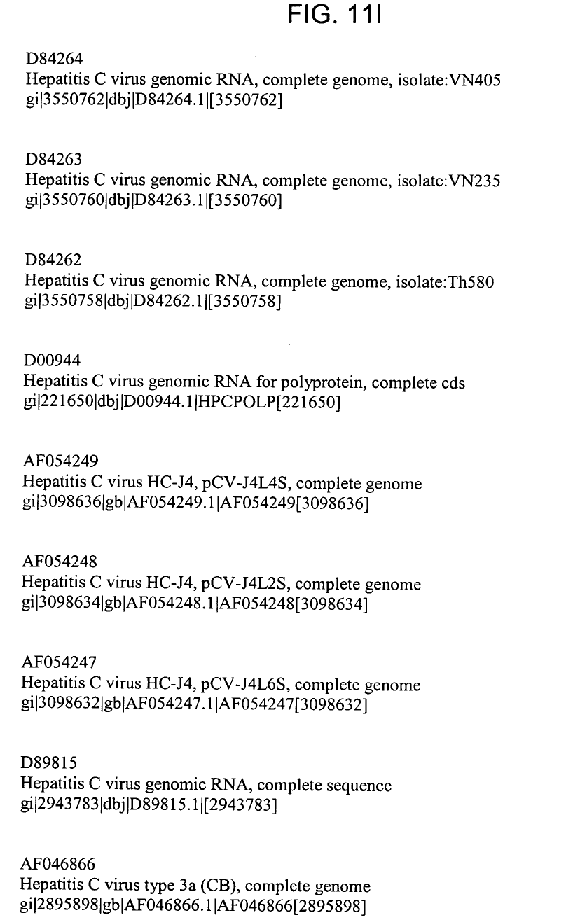

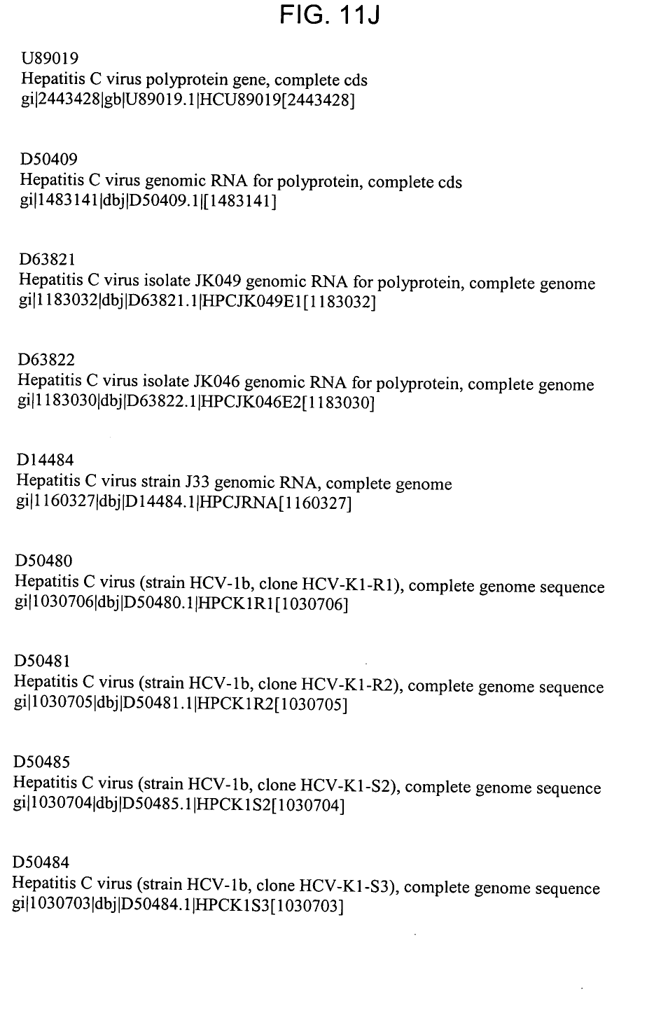

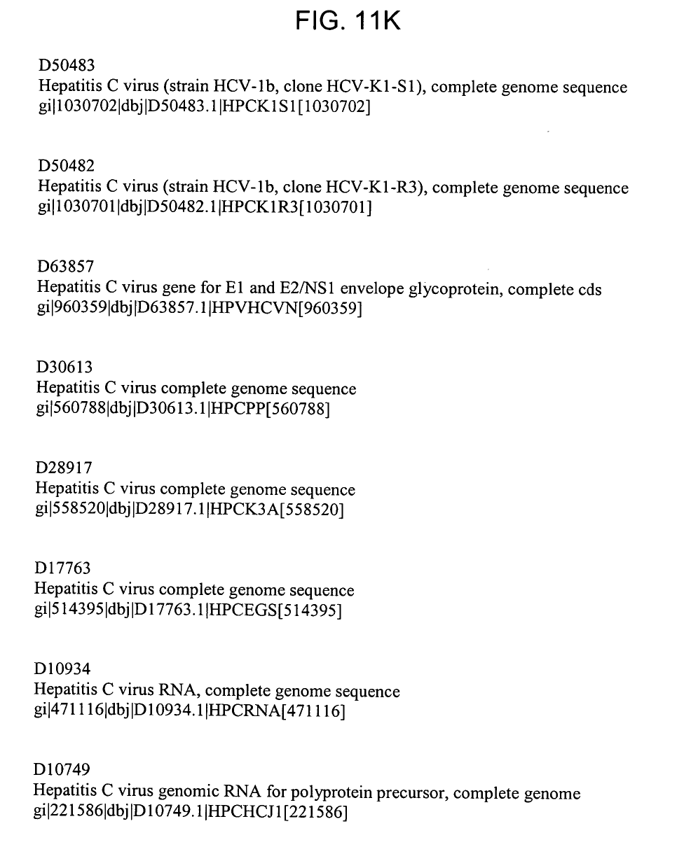

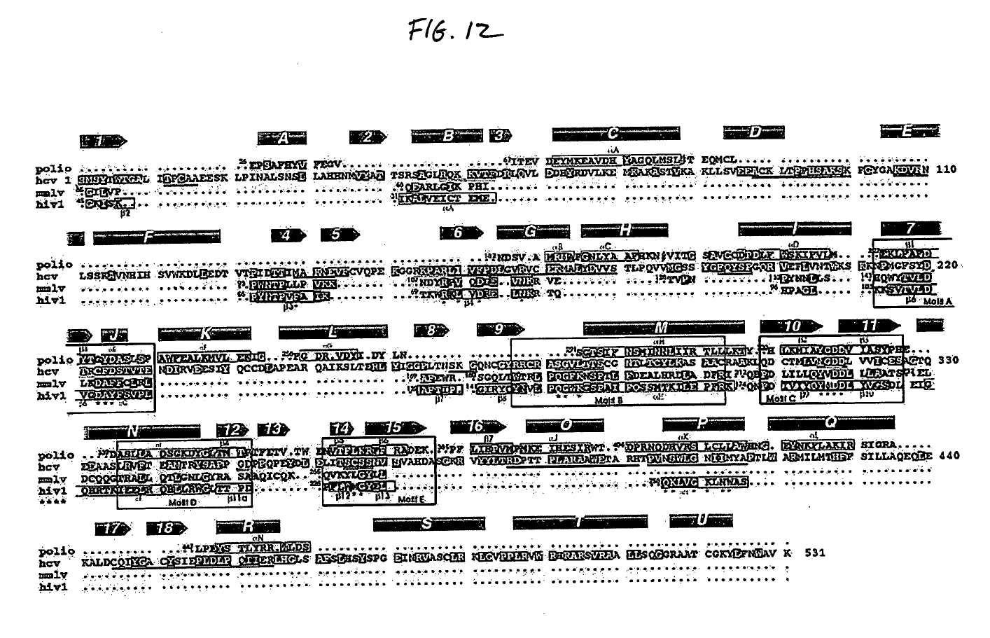

By “HCV” herein is meant any one of a number of different genotypes and isolates of hepatitis C virus. Suitable NS5B polymerases are found in the following HCV isolates: H77 isolate, Chiron isolate, J6 isolate, Con1 isolate, isolate 1; isolate BK; isolate EC1; isolate EC10; isolate HC-J2; isolate HC-J5; isolate HC-J6; isolate HC-J7; isolate HC-J8; isolate HC-JT; isolate HCT18; isolate HCT27; isolate HCV-476; isolate HCV-KF; isolate Hunan; isolate Japanese; isolate Taiwan; isolate TH; isolate type 1; isolate type 1a; Isolate strain H77; Isolate type 1b; Isolate type 1c; Isolate type 1d; Isolate type 1e; Isolate type 1f; Isolate type 10; Isolate type 2; Isolate type 2a; Isolate type 2b; Isolate type 2c; Isolate type 2d; Isolate type 2f; Isolate type 3; Isolate type 3a; Isolate type 3b; Isolate type 3g; Isolate type 4; Isolate type 4a; Isolate type 4c; Isolate type 4d; Isolate type 4f; Isolate type 4h; Isolate type 4k; Isolate type 5; Isolate type 5a; Isolate type 6; and Isolate type 6a. FIGS. 11A-K depict the Genbank accession numbers for a number of HCV genomes, from which the NS5B sequences are easily determined for use in the inventions described herein. As will be appreciated by those in the art, these are nucleic acids encoding the NS5B polymerase, with the latter being the focus of the various assays and methods described herein. “Bioactive agent” or “active agent” refers to an agent, generally selected from a population or library of candidate bioactive agents, defined below, that shows an effect on at least one biochemical activity of an HCV NS5B polymerase, as discussed below. In general, bioactive agents are those which exhibit IC₅₀s in the particular assay in the range of about 1 mM or less. Compounds which exhibit lower IC₅₀s, for example, in the range of about 100 μM, 10 μM, 1 μM, 100 nM, 10 nM, 1 nM, or even lower, are particularly useful for as therapeutics or prophylactics to treat or prevent HCV infections, and thus assays which result in these IC₅₀s are preferred. Alternatively, active compounds are those which exhibit an LD₅₀ (i.e., concentration of compound that kills 50% of the virus) in the range of about 1 mM or less. Compounds which exhibit a lower LD₅₀, for example, in the range of about 100 μM, 10 μM, 1 μM, 100 nM, 10 nM, 1 nM, or even lower, are particularly useful for as therapeutics or prophylactics to treat or prevent HCV infections.

“Candidate bioactive agent” or “candidate drug” as used herein describes any molecule, e.g., protein, oligopeptide, small organic molecule, polysaccharide, nucleic acid, etc. that can be screened for activity as outlined herein. Candidate agents encompass numerous chemical classes, though typically they are organic molecules, preferably small organic compounds having a molecular weight of more than 100 and less than about 2,500 daltons. Particularly preferred are small organic compounds having a molecular weight of more than 100 and less than about 2,000 daltons, more preferably less than about 1500 daltons, more preferably less than about 1000 daltons, more preferably less than 500 daltons. Candidate agents comprise functional groups necessary for structural interaction with proteins, particularly hydrogen bonding, and typically include at least one of an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression and/or synthesis of randomized oligonucleotides and peptides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In one preferred embodiment, the candidate agents are antibodies, a class of proteins. The term “antibody” includes full-length as well antibody fragments, as are known in the art, including Fab, Fab₂, single chain antibodies (Fv for example), chimeric antibodies, etc., either produced by the modification of whole antibodies or those synthesized de novo using recombinant DNA technologies.

“Label” as used herein refers to a detectable moiety. As will be appreciated by those in the art, suitable labels for use in the screening methods of the invention encompass a wide variety of possible moieties. In general, labels include, but are not limited to, a) isotopic labels, which may be radioactive or heavy isotopes; b) immune labels, which may be antibodies or antigens; c) optical dyes, including colored or fluorescent dyes, ) enzymes such as alkaline phosphotase and horseradish peroxidase, e) particles such as colloids, magnetic particles, etc. Preferred labels include chromophores or phosphors but are preferably fluorescent dyes. Suitable dyes for use in the invention include, but are not limited to, fluorescent lanthanide complexes, including those of Europium and Terbium, fluorescein, rhodamine, tetramethylrhodamine, eosin, erythrosin, coumarin, methyl-coumarins, quantum dots (also referred to as “nanocrystals”), pyrene, Malacite green, stilbene, Lucifer Yellow, Cascade Blue™, Texas Red, Cy dyes (Cy3, Cy5, etc.), alexa dyes, phycoerythin, bodipy, and others described in the 6th Edition of the Molecular Probes Handbook by Richard P. Haugland, hereby expressly incorporated by reference.

In some embodiments, an NS5B polymerase can be labeled as a fusion protein with an autofluorescent protein. In one embodiment, the autofluorescent protein is a green fluorescent protein (GFP). In a specific embodiment, the autofluorescent protein is a GFP from Aequorea, or one of the well-known variants thereof including red fluorescent protein (RFP), blue fluorescent protein (BFP), and yellow fluorescent protein (YFP). In another specific embodiment, the autofluorescent protein is a GFP from a Renilla species. In another specific embodiment, the autofluorescent protein is a GFP from Ptilosarcus. In another specific embodiment, the autofluorescent protein is a GFP homologue from Anthozoa species (Matz et al., Nat. Biotech., 17:969-973, 1999).

Included within the definition of labels are FRET labels. As is known in the art, FRET labels in close spatial proximity allow fluorescence resonance energy transfer (FRET). That is, the excitation spectra of the first FRET label overlaps the emission spectra of the second FRET label. Accordingly, exciting the first label results in second label emission.

“Library” refers to at least two compounds. In the context of using libraries of different candidate bioactive agents, the library preferably should provide a sufficiently structurally diverse population of randomized, biased or targeted candidate agents to effect a probabilistically sufficient range of diversity to allow binding to a particular target, in this case the pocket region of the HCV NS5B polymerase.

“Nucleic acid” or “oligonucleotide” or grammatical equivalents refers to at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp 169-176). Several nucleic acid analogs are described in Rawls, C & E News Jun. 2, 1997 page 35. All of these references are hereby expressly incorporated by reference. These modifications of the ribose-phosphate backbone may be done to facilitate their use as candidate agents or inhibitors, or to increase the stability and half-life of such molecules in physiological environments.

As will be appreciated by those in the art, all of these nucleic acid analogs may find use in the present invention. In addition, mixtures of naturally occurring nucleic acids and analogs can be made. Alternatively, mixtures of different nucleic acid analogs, and mixtures of naturally occurring nucleic acids and analogs may be made.

The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xanthine hypoxanthine, isocytosine, isoguanine, etc. As used herein, the term “nucleoside” includes nucleotides as well as nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as a nucleoside.

In some cases, the candidate agent is an RNA molecule, including RNA analogs, that are labeled, to test for binding to the pocket region.

“Proteins” or grammatical equivalents herein refers to proteins, oligopeptides and peptides, derivatives and analogs, including proteins containing non-naturally occurring amino acids and amino acid analogs, and peptidomimetic structures. The side chains may be in either the (R) or the (S) configuration. In a preferred embodiment, the amino acids are in the (S) or L-configuration.

Similarly, a “recombinant protein” is a protein made using recombinant techniques, i.e. through the expression of a recombinant nucleic acid as depicted above. A recombinant protein is distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises at least about 75% by weight of the total protein, with at least about 80% being preferred, and at least about 90% being particularly preferred. The definition includes the production of an NS5B polymerase from one organism in a different organism or host cell. Alternatively, the protein may be made at a significantly higher concentration than is normally seen, through the use of a inducible promoter or high expression promoter, such that the protein is made at increased concentration levels. Alternatively, the protein may be in a form not normally found in nature, as in the addition of an epitope tag or amino acid substitutions, insertions and deletions, as discussed below.

“Solid support” or “substrate” or other grammatical equivalents herein is meant any material that can be utilized in the heterogeneous assays systems outlined below. In general, the support will be amenable to the detection system of choice (e.g. fluorescence when fluors are used as the label, surface plasmon resonance assays, etc.). Suitable substrates include metal surfaces such as gold, glass and modified or functionalized glass, fiberglass, teflon, ceramics, mica, plastic (including acrylics, polystyrene and copolymers of styrene and other materials, polypropylene, polyethylene, polybutylene, polyimide, polycarbonate, polyurethanes, Teflon™, and derivatives thereof, etc.), GETEK (a blend of polypropylene oxide and fiberglass), etc, polysaccharides, nylon or nitrocellulose, resins, silica or silica-based materials including silicon and modified silicon, carbon, metals, inorganic glasses and a variety of other polymers. Particularly preferred solid supports are those that allow high throughput screening, such as microtiter plates and beads (sometimes referred to herein as microspheres). The composition of the beads will vary, depending on the use. Suitable bead compositions include those used in peptide, nucleic acid and organic moiety synthesis, including, but not limited to, plastics, ceramics, glass, polystyrene, methylstyrene, acrylic polymers, paramagnetic materials, thoria sol, carbon graphite, titanium dioxide, latex or cross-linked dextrans such as Sepharose, cellulose, nylon, cross-linked micelles and Teflon may all be used. “Microsphere Detection Guide” from Bangs Laboratories, Fishers, Ind. is a helpful guide.

6.2 Description of the Preferred Embodiments

The present disclosure is directed to the discovery that several novel classes of compounds, identified as inhibitors of HCV replication, associate with amino acid residues in a particular pocket, called the “Rigel pocket,” of the NS5B RNA dependent RNA polymerase of the HCV virus. Although this pocket has been speculated to be involved with certain essential biological functions, including the oligomerization of the NS5B polymerase, the interaction of the NS5B polymerase with other HCV proteins, and the RNA binding domain, (the latter domain based on structural analogy to the HIV reverse transcriptase protein), this pocket has never before been identified as the target for HCV inhibitory compounds. Moreover, certain amino acid residues within this pocket have been identified as likely points of contact, interaction and/or association for such HCV inhibitory compounds. These residues have never before been identified as important points of contact, interaction and/or association for HCV inhibitory compounds.

Taken together, the binding of this class of inhibitors (referred to herein as “pocket binding inhibitors”, or PBIs) is responsible for potently inhibiting HCV replication. In addition to allowing for novel methods of both inhibiting the HCV NS5B polymerase and methods of treating HCV infections, the discovery of the mechanism of action (MOA) allows the design of a wide variety of screening methods, both biochemical assays and in silico assays, to elucidate additional agents active in the inhibition of HCV infection and/or replication, allowing for the discovery of further PBIs.

Accordingly, the present disclosure is drawn to methods of inhibiting an HCV NS5B polymerase which comprises contacting the polymerase with a bioactive agent that binds to the Rigel pocket. As is more fully described below, any molecule that binds to the Rigel pocket, including the compounds described herein and in the incorporated applications, can be used either to inhibit NS5B polymerase, to inhibit HCV replication and/or proliferation, to treat or prevent HCV infections, or in the assays described below to elucidate additional inhibitors.

The methods described herein are directed to the inhibition of HCV NS5B polymerases. The term “NS5B” or “NS5B polymerase” refers to an HCV RNA dependent RNA polymerase. Depending on the particular application, the NS5B polymerases from any wild-type (or in some cases derivative proteins, as outlined below) can be used. In general, recombinant or isolated NS5B polymerases, as defined below, are used in screening assays as defined below.

A recent report based on crystallographic studies shows that one class of HCV NS5B inhibitors, which are phenylalanaine derivatives, bind to a binding site in the “thumb subdomain” near the C terminus of HCV NS5B polymerase. See Wang et al., 2003, J. Biol. Chem. 278:9489 the disclosure of which is incorporated herein by reference. These derivatives, and others that bind within the same domain, will be referred to herein as the “thumb subdomain inhibitors”, or TSIs. The methods outlined herein are designed to use and/or elucidate PBIs and not TSIs; thus TSIs are not preferred in most cases, except for use in combination therapies with PBIs.

Thus in certain aspects, the present disclosure is directed to a variety of assays that permit the design or identification of molecules that specifically bind in the pocket region and not in the thumb subdomain. In particular, a variety of competition assays that rely on the use of these previously identified PBIs are preferred.

As used herein, a polymerase is a “NS5B polymerase” if the overall homology of the amino acid sequence to the amino acid sequences of a known NS5B polymerase, e.g., the NS5B polymerases contained within Genbank accession numbers AJ238799 (amino acid residues 2421-3011; nucleotides 7599-9371) or M62321 (amino acid residues 2421-3011)), is greater than about 70%, preferably greater than about 75%, more preferably greater than about 80%, even more preferably greater than about 85% and most preferably greater than 90%. In some embodiments the homology will be as high as about 93%, 94%, 95%, 96%, 97%, 98%, 99% or even higher. Homology in this context means sequence similarity or identity, with identity being preferred. This homology can be determined using standard techniques known in the art, including, but not limited to, the local homology algorithm of Smith & Waterman, 1981, Adv. Appl. Math. 2:482; the homology alignment algorith of Needleman & Wunsch, 1970, J. Mol. Biool. 48:443; the search for similarity method of Pearson & Lipman, 1988, Proc. Natl. Acad. Sci. USA 85:2444; computerized implementations of these algorithms (GAP, BESTFIT, FASTA, and TFASTA in the Wisconsin Genetics Software Package, Genetics Computer Group, 575 Science Drive, Madison, Wis.); the Best Fit sequence program described by Devereux et al., 1984, Nucl. Acid Res. 12:387-395, preferably using the default settings, or by inspection.

One example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, 1987, J. Mol. Evol. 35:351-360; the method is similar to that described by Higgins & Sharp, 1989, CABIOS 5:151-153. Useful PILEUP parameters including a default gap weight of 3.00, a default gap length weight of 0.10, and weighted end gaps.

Another example of a useful algorithm is the BLAST algorithm, described in Altschul et al., 1990, J. Mol. Biol. 215, 403-410 and Karlin et al., 1993, Proc. Natl. Acad. Sci. USA 90:5873-5787. A particularly useful BLAST program is the WU-BLAST-2 program which was obtained from Altschul et al., 1996, Methods in Enzymology, 266:460-480; ://blast.wustl/edu/blast/ README.html]. WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity. A % amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored). The alignment may include the introduction of gaps in the sequences to be aligned.

NS5B polymerases may be shorter or longer than the naturally occurring amino acid sequences. Thus, included within the definition of NS5B polymerases are portions or fragments of the sequences depicted herein. Fragments of NS5B polymerases are considered NS5B polymerases if a) they exhibit the ability to bind a PBI; b) have at least the indicated homology; and c) and preferably have at least one NS5B biological activity. In addition, certain embodiments include polymerases that share at least one antigenic epitope with a naturally occurring NS5B polymerase, although in many instances this many not be required.

In addition, in particular for use in in silico assays, it is possible to use the structural coordinates for discontinuous residues, e.g. those defining the pocket region, rather than a linear fragment. Discontinuous regions that contribute to the pocket region are described below, and any, all or combinations thereof may find use in the in silico regions.

Also included within the definition of NS5B polymerases are amino acid sequence variants. These variants fall into one or more of three classes: substitutional, insertional or deletional variants. These variants ordinarily are prepared by site specific mutagenesis of nucleotides in the DNA encoding the NS5B polymerase, using cassette or PCR mutagenesis or other techniques well known in the art, to produce DNA encoding the variant, and thereafter expressing the DNA in recombinant cell culture as outlined above. However, variant NS5B polymerase fragments having up to about 100-150 residues may be prepared by in vitro synthesis using established techniques. Amino acid sequence variants are characterized by the predetermined nature of the variation, a feature that sets them apart from naturally occurring allelic or interspecies variation of the NS5B polymerase amino acid sequence. The variants typically exhibit the same qualitative biological activity as the naturally occurring analogue, although variants can also be selected which have modified characteristics as will be more fully outlined below.

While the site or region for introducing an amino acid sequence variation is predetermined, the mutation per se need not be predetermined. For example, in order to optimize the performance of a mutation at a given site, random mutagenesis may be conducted at the target codon or region and the expressed NS5B variants screened for the optimal combination of desired activity. Techniques for making substitution mutations at predetermined sites in DNA having a known sequence are well known, for example, M13 primer mutagenesis and PCR mutagenesis. Screening of the mutants is done using assays of NS5B polymerase activities.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final variant. Generally these changes are done on a few amino acids to minimize the alteration of the molecule. However, larger changes may be tolerated in certain circumstances. When small alterations in the characteristics of the NS5B polymerase are desired, substitutions are generally made in accordance with the following chart:

CHART I

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Substantial changes in function or immunological identity are made by selecting substitutions that are less conservative than those shown in Chart I. For example, substitutions may be made which more significantly affect: the structure of the polypeptide backbone in the area of the alteration, for example the alpha-helical or beta-strand structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine.

The variants typically exhibit the same qualitative biological activity and will elicit the same immune response as the naturally-occurring analogue, although variants also are selected to modify the characteristics of the NS5B polymerases as needed. Alternatively, the variant may be designed such that the biological activity of the NS5B polymerase is altered.

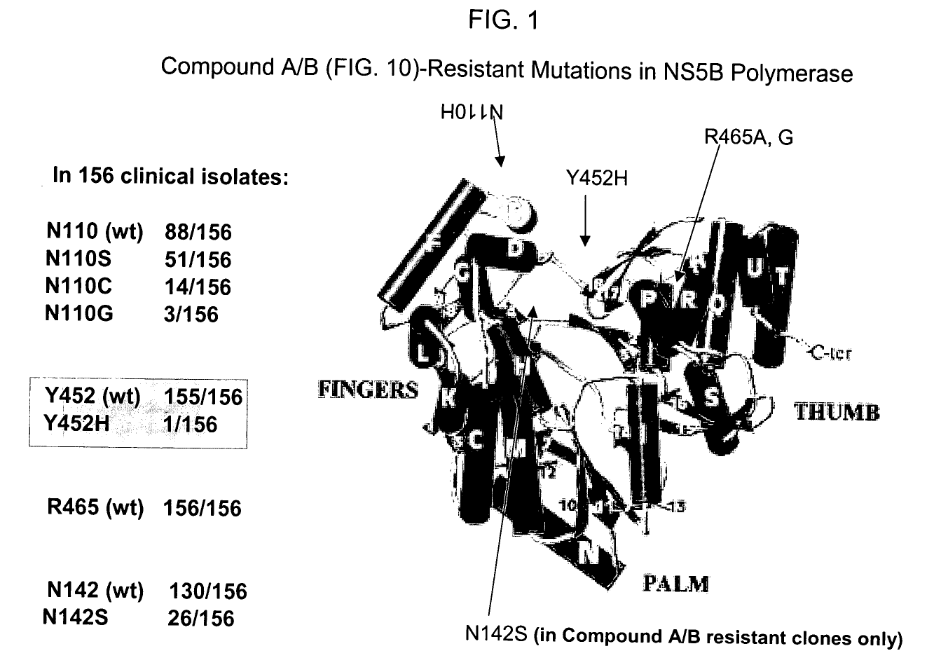

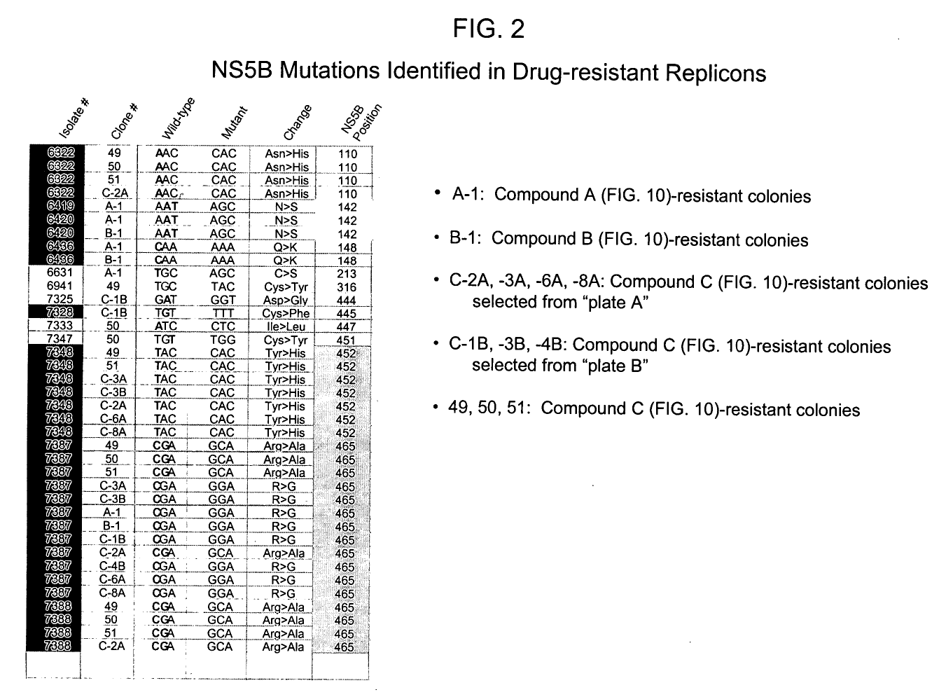

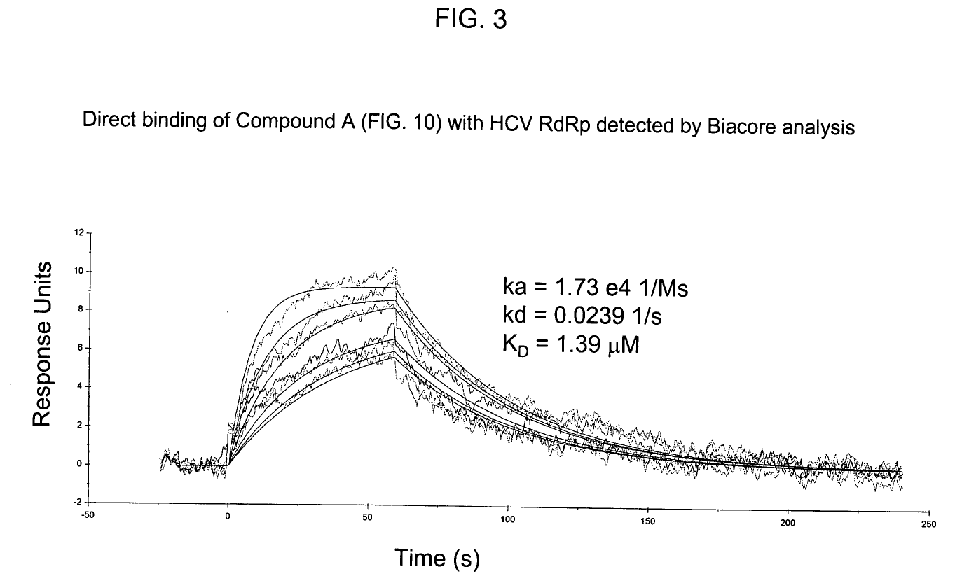

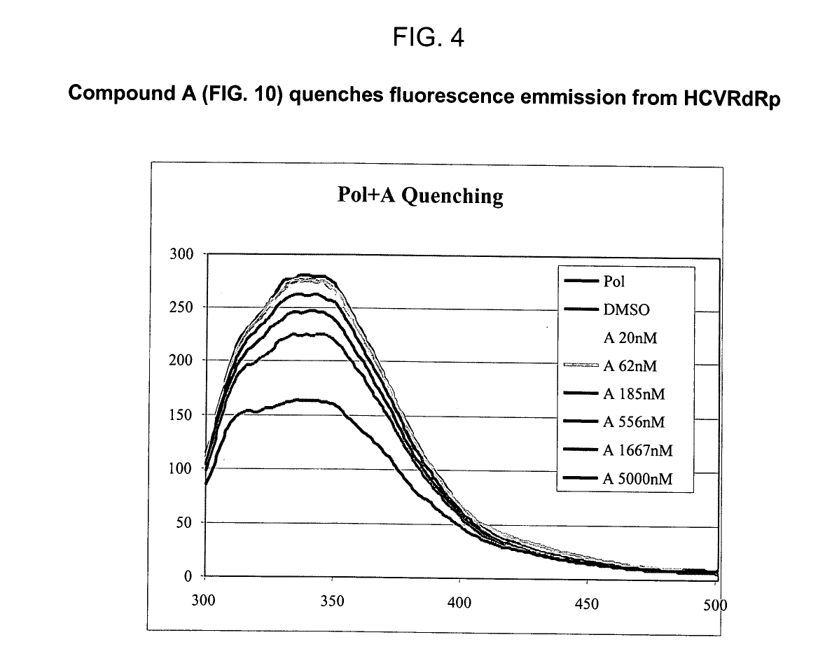

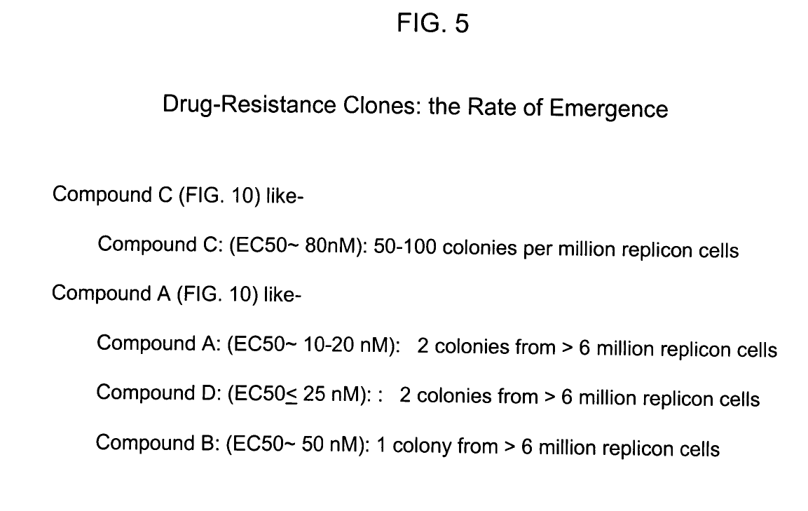

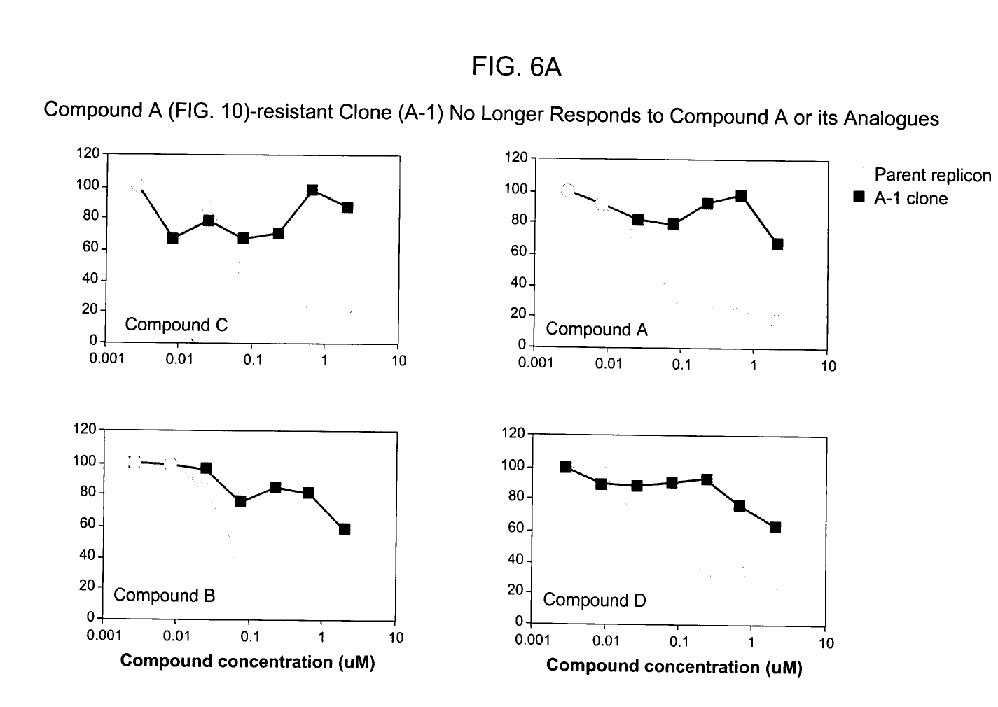

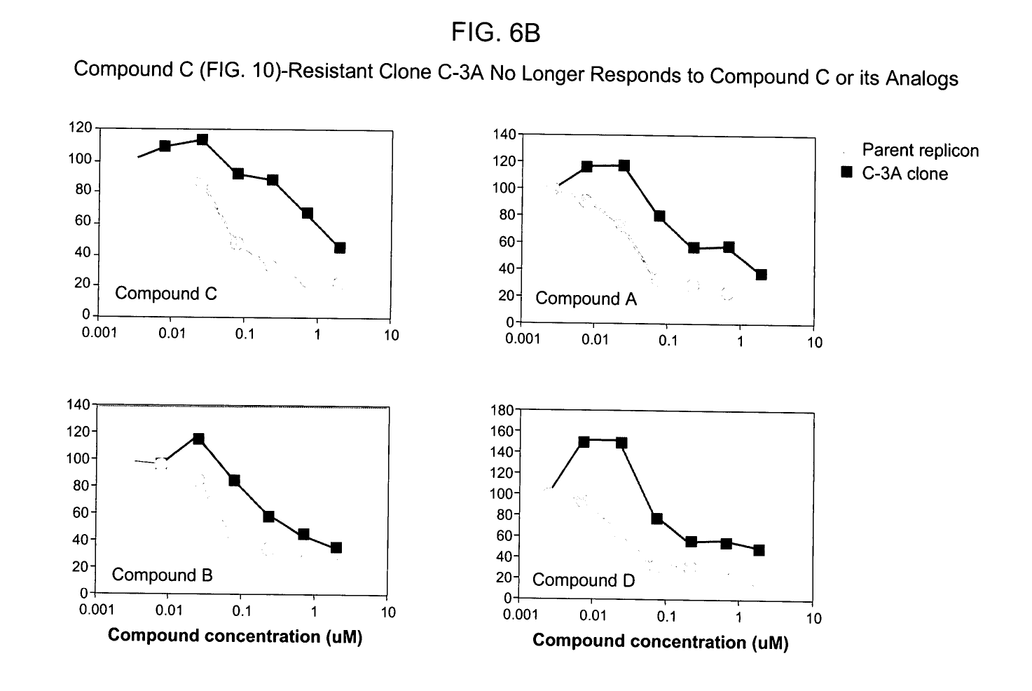

Particularly preferred variants of NS5B correspond to substitutions of one or more residues within the Rigel pocket, as defined below. These variants, particularly variants at positions that are conserved among all or most known HCV genotypes, find particular use in counterscreens to confirm the agent binds to the Rigel pocket; that is, as described below, a loss of inhibitory activity against compounds active against wild-type NS5B polymerase (or variants if the variation is outside the pocket region) will be seen when a variant in an important pocket region is used. In particular, variants within the alpha helical regions depicted as “P”, “O” and “R” and/or the beta strands depicted as “5,” “17” and “18” (and the loops that connect strands “17” and “18”) as shown in instant FIG. 12 and FIG. 2 of Bressanelli et al., 1999, Proc. Natl. Acad. Sci. USA 96:13034-13039, are preferred, particularly in the case of conserved residues. These variants have substitutions independently selected from residue positions 142, 148, 213, 316, 444, 445, 447, 451, 452 and 465, or combinations thereof. Particularly preferred are variants at positions 445, 451, 452 and/or 465, with the latter being especially preferred. Particularly preferred substitutions are shown in instant FIG. 2. These specific amino acid substitutions occur in a defined structural pocket mapped on the surface of the HCV NS5B polymerase (FIG. 1 and described below). Moreover, particular mutations (R465A,G; Y452H; N110H, and N142S) resulted from the drug selection, since they were rarely, if ever, found in published HCV variants of the existing six HCV genotypes. In particular, variants at position 465 that remove a positive charge appear particularly preferred. Since these highly conserved residues are included in the pocket region binding site of the inhibitors, drugs in this class and those discovered using the methods of the invention that associate with this position, and other residues conserved in all genotypes will be effective in inhibiting HCV of all the genotypes.

Moreover, these variants may be used to define or identify useful PBI compounds or classes of PBI compounds (defined below). As mentioned above, mutations at these positions are not found in nature. Rather, they were introduced into the NS5B polymerase by the particular PBI compounds indicated in FIG. 2 during replicon selection assays. As variants including these mutations are resistant to treatment with PBIs, it is presumed that the residues at these positions, as well as the 1-3 or so residues flanking these positions, are essential for plymerase activity. Thus, PBI compounds may also be defined based upon their ability to induce mutations in an NS5B polymerase at one or more of the positions discussed above. PBI compounds having such properties are potent inhibitors of the NS5B polymerase and HCV replication, and are therefore useful in the treatment or prevention of HCV.

Covalent modifications of NS5B polymerases are included within the scope of this invention, particularly for screening assays. One type of covalent modification includes reacting targeted amino acid residues of an NS5B polypeptide with an organic derivatizing agent that is capable of reacting with selected side chains or the N- or C-terminal residues of an NS5B polypeptide. Derivatization with biflnctional agents is useful, for instance, for crosslinking NS5B to a water-insoluble support matrix or surface for use in the methods described below. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunonal imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the “-amino groups of lysine, arginine, and histidine side chains (T. E. Creighton, Proteins: Structure and Molecular Properties, W.H. Freeman & Co., San Francisco, pp. 79-86 (1983)), acetylation of the N-terminal amine, and amidation of any C-terminal carboxyl group.

NS5B polymerases may also be modified in a way to form chimeric molecules comprising an NS5B polypeptide fused to another, heterologous polypeptide or amino acid sequence. In one embodiment, such a chimeric molecule comprises a fusion of an NS5B polypeptide with a tag polypeptide which provides an epitope to which an anti-tag antibody can selectively bind. The epitope tag is generally placed at the amino-or carboxyl-terminus of the NS5B polypeptide (or it may be added to the “new” C-terminus after the hydrophobic amino acid region, generally about 21 residues, is removed). The presence of such epitope-tagged forms of an NS5B polypeptide can be detected using an antibody against the tag polypeptide. Also, provision of the epitope tag enables the NS5B polypeptide to be readily purified by affinity purification using an anti-tag antibody or another type of affinity matrix that binds to the epitope tag; this is also useful for binding the protein to a support for heterogeneous screening methods. Various tag polypeptides and their respective antibodies are well known in the art. Examples include poly-histidine (poly-his) or poly-histidine-glycine (poly-his-gly) tags; the flu HA tag polypeptide and its antibody 12CA5 (Field et al., 1998, Mol. Cell. Biol. 8:2159-2165; the c-myc tag and the 8F9, 3C7, 6E10, G4, B7 and 9E10 antibodies thereto (Evan et al., 1985, Molecular and Cellular Biology 5:3610-3616); and the Herpes Simplex virus glycoprotein D (gD) tag and its antibody (Paborsky et al., 1990, Protein Engineering 3(6):547-553). Other tag polypeptides include the Flag-peptide (Hopp et al., 1988, BioTechnology 6:1204-1210); the KT3 epitope peptide (Martin et al., 1992, Science 255:192-194); tubulin epitope peptide (Skinner et al., 1991, J. Biol. Chem. 266:15163-15166); and the T7 gene 10 protein peptide tag (Lutz-Freyermuth et al., 1990, Proc. Natl. Acad. Sci. USA 87:6393-6397). Particularly preferred fusions, particularly for the purposes of screening for PBI compounds, will have fusions (including internal fusions) to areas of the protein distant from the pocket domain. For example, the external loops of the “palm” region may be used for inclusion fusions of tags.

Of paramount importance to the methods of the invention is the identification of the Rigel pocket region of the NS5B polymerase, which serves to define the mechanism of action of the inhibitors described herein and in the incorporated applications, and in addition serves as a focus for the assays described herein.

Referring to instant FIGS. 1 and 12 and FIGS. 1 and 2 of Bressanelli et al., 1999, Proc. Natl. Acad. Sci USA 96:13034-13039, incorporated herein by reference, the “Rigel pocket region” or “pocket region” or “pocket” is defined by the flap region of the NS5B polymerase (beta strands “17” and “18” of Bressanelli, supra and the loop that connects them) and the alpha helices designated as “O”, “P” and “R” of Bressanelli, supra, that reside in the thumb subdomain of the NS5B polymerase. With reference to FIG. 2 of Bressanelli, supra, these various structural regions are approximately defined by residues 389-466 of the NS5B polymerase (plus or minus 1-2 residues on either or both ends). Additional definition may be provided by beta strand “5.” The approximate residues defining these structural elements (plus or minus 1-2 residues on either or both ends) are as follows:

| Structural Element | Residues |
|---|---|
| beta strand “5” | 142-147 |
| beta strand “17” | 442-447 |
| beta strand “18” | 450-454 |
| loop connecting “17” with “18” | 448-449 |
| alpha helix “O” | 389-398 |
| alpha helix “P” | 406-416 |
| alph helix “R” | 459-466 |

As will be recognized by skilled artisans, while all of these structural elements are believed to define the Rigel pocket, they may not all be necessary. At a minimum, it is believed that the beta strands “17” and “18” of the flap region and the alpha helix “R” define the pocket. Thus, at a minimum, residues 442-466 (plus or minus 1-2 resides on either or both ends) are believed to define the pocket. Alpha helix “P” may provide additional definition. Thus, in some embodiments, the pocket may be defined by residues 405-466 (plus or minus 1-2 residues on either or both ends).

Empirical data obtained from PBI-resistant mutants indicates that specific residues within the structural elements defining the Rigel pocket may play important roles in defining the pocket. These residues include, but are not limited to, the residues at positions 142, 148, 213, 316, 444, 445, 447, 451, 452, and 465, as numbered according to FIG. 2 of Bressanelli, supra. Similar residues in NS5B polymerases from different HCV strains are easily identified as is known in the art.

Among all the mutated residues shown in the instant FIGS, Arg-465 appears crucial as at least one of the direct binding sites for PBIs. This is based on the observation that Arg-465 was mutated to either a glycine or an alanine in each and all of the analyzed resistant clones (FIG. 2). Interestingly, Arg-465 maps to the so called “Armadillo repeats” on the HCV NS5B polymerase, and the Arm-repeats are known to be involved in protein-protein interactions. It is known that the formation of the replicase complex involves a lot of interaction between the NS5B polymerase and other viral and host proteins.

Other residues believed to be particularly important in defining the pocket and which may constitute points of contact, association or interaction with PBI compounds include Tyr-452 and optionally Cys-445 and Cys-451. These latter two Cys residues are believed to be important in maintaining the correct conformation of the flap region of the NS5B polymerase.

The Rigel pocket region is involved in dynamic conformational changes that are required for the enzymatic activity of the HCV NS5B polymerase, including the oligomerization of the polymerase, and in the interaction of the polymerase with other HCV proteins. Furthermore, it appears that the pocket is a part of the RNA-binding domain based on the structural analogy to the HIV reverse transcriptase. These activities are essential for the assembly of the functional, multipartite HCV replication complex.

“Pocket binding inhibitor” or “PBI” refers to any compound that binds to the Rigel pocket region of an HCV NS5B polymerase as defined above and that inhibits at least one biochemical activities of the polymerase as defined herein. The PBI may interact with, associate with and/or contact one or a plurality of residues that define the pocket. The contacts, associations and/or interactions may be any of the types of contacts, associations, or interactions commonly made between binding molecules, ranging from hydrogen binds, to ionic bond or salt bridges, to electrostatic, hydrophobic or van der Waals interactions. Typically, such interactions will not be covalent, although in the case of a suicide PBI, covalent interactions may be observed. Thus, in general, a PBI may contact, associate with and/or interact with one or more residues residing any of the regions, or any combinations of such regions, defining the pocket. Accordingly, in one embodiment, the PBI contacts, interacts, binds to and/or associates with a residue residing within positions 389-466 of the NS5B polymerase (using the numbering of FIG. 2 of Bressanelli, supra). Specific embodiments utilize PBIs that interact with residues within the “R”, “O”, “P”, “5,” “17” and/or “18” regions, either independently or in any combination.

In an additional specific embodiment, the PBIs contact, interact, bind to and/or associate with a residue of NS5B polymerase selected from the group of consisting of positions 142, 148, 213, 316, 44, 445, 447, 451, 452 and 465, either independently or in any combination, with PBIs that contact, interact, bind to and/or associate with the residues at one or both of positions 452 and 465 being particularly preferred.

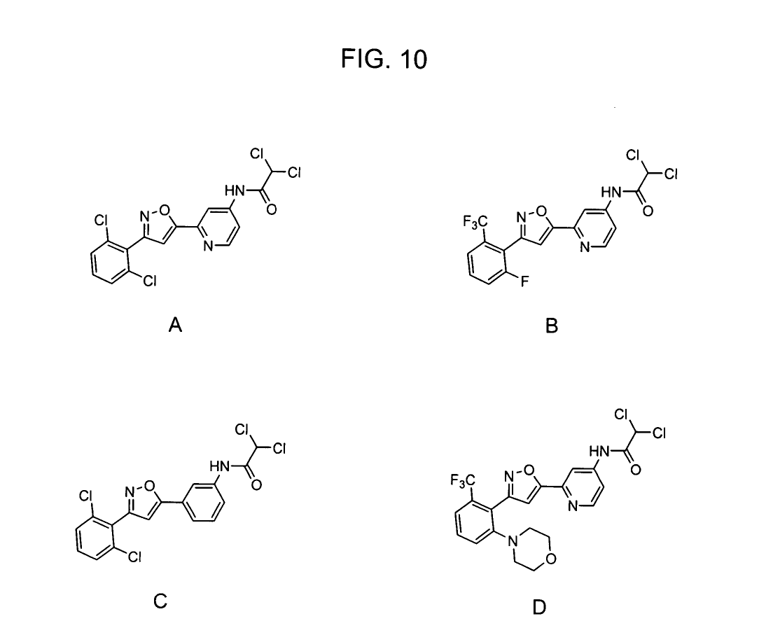

Without intending to be limited by any theory of operation, it is believed that the dichloroacetamide group of the classes of PBI compounds exemplified by the specific structures illustrated in FIG. 10 contacts or otherwise interacts with the side chain of Arg⁴⁶⁵. The remainder of the molecule is believed to be positioned such that it is bounded by the other structural elements defining the pocket. This positioning and/or site of contact or interaction may be used as a guiding tool in the various in silico screening and design methods described in more detail in a later section.

PBI compounds generally comprise two six membered substituted aryl or heteroaryl rings joined by a substituted or unsubstituted 5 membered carbocyclic or heterocyclic ring which may be saturated, unsaturated or aromatic. In some embodiments, PBI compounds include compounds according to structural formulae (I)-(XII): 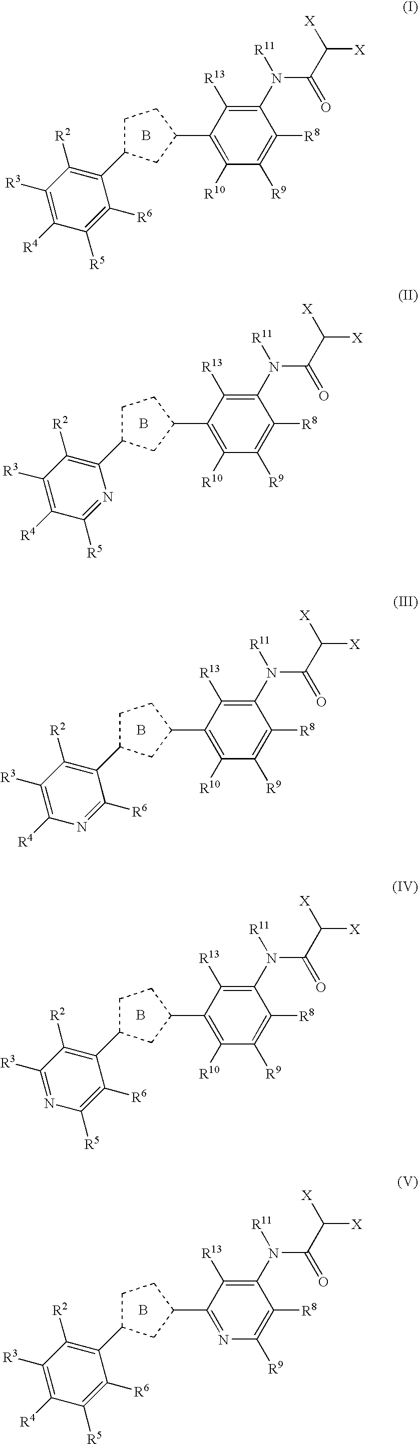 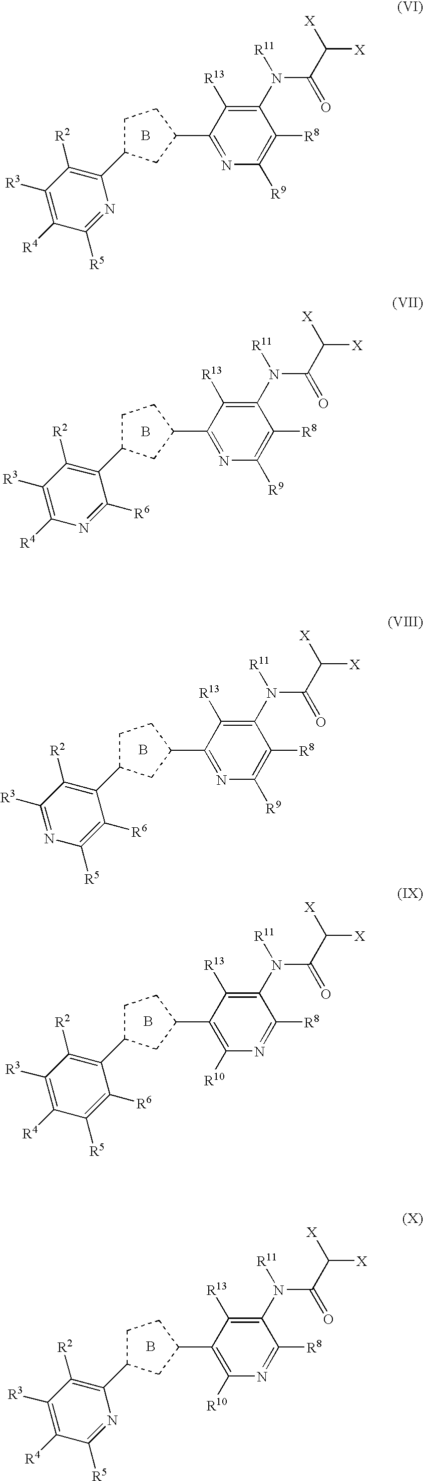 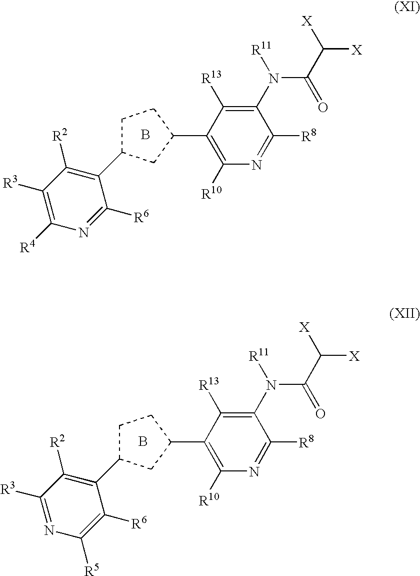
and the salts, hydrates, solvates and oxides thereof, wherein:
“B” represents a five-membered saturated, unsaturated or aromatic ring containing from one to four heteroatoms selected from N, (or NH), O and S, with the proviso that in rings containing two O atoms, the O atoms are not positioned adjacent to one another;
each “X” independently represents a halo group;
R² and R⁶ are each, independently of one another, selected from the group consisting of hydrogen, halo, fluoro, chloro, alkyl, methyl, substituted alkyl, alkylthio, substituted alkylthio, alkoxy, methoxy, i-propoxy, substituted alkoxy, alkoxycarbonyl, substituted alkoxycarbonyl, arylalkyloxycarbonyl, substituted arylalkyloxycarbonyl, aryloxycarbonyl, substituted aryloxycarbonyl, cycloheteroalkyl, substituted cycloheteroalkyl, carbamoyl, substituted carbamoyl, haloalkyl, trifluromethyl, sulfamoyl, substituted sulfamoyl and silyl ether, provided that at least one of R² or R⁶ is other than hydrogen;
R³ and R⁵ are each, independently of one another, selected from the group consisting of hydrogen, halo, chloro, alkyl, substituted alkyl, alkylthio, substituted alkylthio, alkoxy, substituted alkoxy, alkoxycarbonyl, substituted alkoxycarbonyl, arylalkyloxycarbonyl, substituted arylalkyloxycarbonyl, aryloxycarbonyl, substituted aryloxycarbonyl, cycloheteroalkyl, substituted cycloheteroalkyl, carbamoyl, substituted carbamoyl, haloalkyl, sulfamoyl and substituted sulfamoyl;
R⁴ is selected from the group consisting of hydrogen, halo, alkyl, substituted alkyl, alkylthio, substituted alkylthio, carbamoyl, substituted carbamoyl, alkoxy, substituted alkoxy, alkoxycarbonyl, substituted alkoxycarbonyl, arylalkyloxycarbonyl, substituted arylalkyloxycarbonyl, aryloxycarbonyl, substituted aryloxycarbonyl, dialkylamino, substituted dialkylamino, haloalkyl, sulfamoyl and substituted sulfamoyl;
R⁸, R⁹, R¹⁰ and R¹³ are each, independently of one another, selected from the group consisting of hydrogen, halo and fluoro; and
R¹¹ is selected from the group consisting of hydrogen, alkyl and methyl.

When the substituent group defining a particular R², R³, R⁴, R⁵ and/or R⁶ variable is substituted, the nature of the substitution can vary broadly. Non-limiting examples of suitable groups useful for substituting such substituents include (C1-C6) alkyl (linear, branched or cyclic, saturated or unsaturated), —O⁻, ═O, —OR^{a}, —S⁻, ═S, —SR^{a}—NR^{c}R^{c}, ═NR^{a}, —CX₃, —CF₃, —CN, —OCN, —SCN, —NO, —NO₂, ═N₂, —N₃, —S(O)₂R^{a}, —S(O)₂O⁻, —S(O)₂OR^{a}, —OS(O)₂R^{a}, —OS(O₂)O—, —OS(O₂)OR^{a}, —P(O)(O⁻)₂, —P(O)(OR^{a})(O⁻), —OP(O)(OR^{a})(OR^{a}), —C(O)R^{a}, —C(S)R^{a}, —C(O)O⁻, —C(O)OR^{a}, —C(S)OR^{a}, —C(O)NR^{c}R^{c}, —C(NR^{a})NR^{c}R^{c}, —NR^{a}C(O)NR^{c}R^{c}, —NR^{a}C(S)NR^{c}R^{c}, and —NR^{a}C(NR^{a})NR^{c}R^{c} where each R^{a} is independently selected from hydrogen and (C1-C6) alkyl (linear, branched or cyclic, saturated or unsaturated) and each R^{c} is, independently of the other, an R^{a} or, alternatively, two R^{c} groups may be taken together with the nitrogen atom to which they are bonded to form a 3 to 8 membered ring which may optionally include from one to four of the same or different heteroatoms selected from O, S and N (or NH).

The identity of the “B” ring can vary broadly. In some embodiments, the “B” ring is a heterocyclic ring selected from isoxazolyl, pyrazolyl, oxadiazolyl, oxazolyl, thiazolyl, imidazolyl, triazolyl, thiadiazolyl and hydro isomers. Suitable hydro isomers include, but are not limited to, dihydro and tetrahydro isomers of the stated rings. Specific examples of such hydro isomers include, for example, 2-isoxazolinyls, 3-isoxazolinyls, 4-isoxazolinyls, isoxazolidinyls, 1,2-pyrazolinyls, 1,2-pyrazolidinyls, (3H)-dihydro-1,2,4-oxadiazolyls, (5H)-dihydro-1,2,4-oxadiazolyls, oxazolinyls, oxazolidinyls, (3H)-dihydrothiazolyls, (5H)-dihydrothiazolyls, thiazolidinyls (tetrahydrothiazolyls), (3H)-dihydrotriazolyls, (5H)-dihydrotriazolyls, triazolidinyls (tetrahydrotriazolyls), dihydro-oxadiazolyls, tetrahydro-oxadiazolyls, (3H)-dihydro-1,2,4-thiadiazolyls, (5H)-dihydro-1,2,4-thiadiazolyls, 1,2,4-thiadiazolidinyls (tetrahydrothiadiazolyls), (3H)-dihydroimidazolyls, (5H)-dihydroimidazolyls and tetrahydroimidazolyls.

In some embodiments of the PBI compounds according to structural formulae (I)-(XII), the “B” ring is selected from 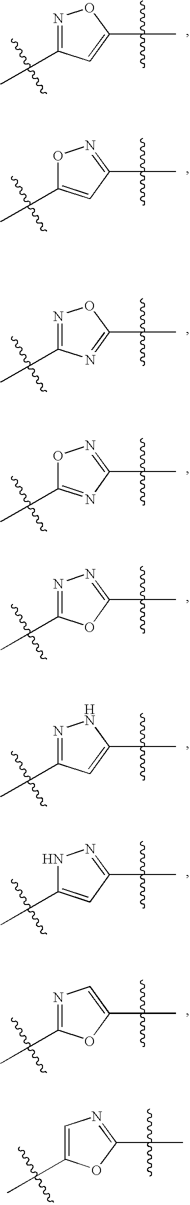
and hydro isomers thereof. In a specific embodiment, the “B” ring is selected from

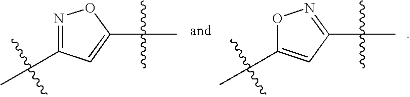

In some embodiments of PBI compounds according to structural formulae (I)-(XII), R² and R⁶ are other than hydrogen and R³, R⁴ and R⁵ are each hydrogen. In one specific embodiment, R² and R⁶ are each, independently of one another, selected from the group consisting of chloro, fluoro, methyl, trifluromethyl, thiomethyl, methoxy, i-propoxy, N-morpholino and N-morpholinosulfamoyl. In another specific embodiment, R² and R⁶ are each, independently of one another, selected from the group consisting of chloro, fluoro, methyl, trifluromethyl, methoxy and i-propoxy. In another specific embodiment, R² and R⁶ are each the same or different halo.

Other embodiments of PBI compounds, as well as specific embodiments of exemplary PBI compounds and methods for their synthesis are described in the various incorporated applications listed in the summary section, above.

Especially preferred embodiments of exemplary PBI compounds are shown in FIGS. 10A, B, C and D.

However, it should be noted that using the methods outlined herein, a variety of other types of inhibitors, including any class described as a “candidate agent”, may be found to be a PBI.

Inhibition of an NS5B polymerase activity can be tested in several ways. For example, inhibition may be assessed using a replicon assay, as is well-known in the art. In the context of treatment, treatment (including amelioration of symptoms, prevention of disease) may be tested as is known in the art, or may be based on anecdotal evidence. Generally, at least a 25% decrease in at least one of the biochemical activities of the NS5B polymerase is preferred, with at least about 50% being particularly preferred and about a 95-100% decrease being especially preferred, and IC₅₀s and LD₅₀s are as outlined above for bioactive agents. See the section of “Modulation of Activity” for specific assays to confirm inhibition.

6.3 Uses and Administration

Owing to their ability to inhibit the NS5B polymerase and HCV replication, PBI compounds and/or compositions thereof can be used in a variety of contexts. For example, the PBI compounds can be used as controls in in vitro assays to identify additional anti HCV compounds having greater or lesser potency. As another example, the PBI compounds and/or compositions thereof can be used as preservatives or disinfectants in clinical settings to prevent medical instruments and supplies from becoming infected with HCV virus. When used in this context, the PBI compounds and/or composition thereof may be applied to the instrument to be disinfected at a concentration that is a multiple, for example 1×, 2×, 3×, 4×, 5× or even higher, of the measured IC₅₀ for the compound.

In a specific embodiment, the PBI compounds and/or compositions can be used to “disinfect” organs for transplantation. For example, a liver or portion thereof being prepared for transplantation can be perfused with a solution comprising a PBI compound of the invention prior to implanting the organ into the recipient. This method has proven successful with lamuvidine (3TC, Epivir®, Epivir-HB®) for reducing the incidence of hepatitis B virus (HBV) infection following liver transplant surgery/therapy. Quite interestingly, it has been found that such perfusion therapy not only protects a liver recipient free of HBV infection (HBV−) from contracting HBV from a liver received from an HBV+ donor, but it also protects a liver from an HBV− donor transplanted into an HBV+ recipient from attack by HBV. The PBI compounds and/or compositions including them may be used in a similar manner prior to organ or liver transplantation.

The PBI compounds and/or compositions thereof find particular use in the treatment and/or prevention of HCV infections in animals and humans. When used in this context, the compounds may be administered per se, but are typically formulated and administered in the form of a pharmaceutical composition. The exact composition will depend upon, among other things, the method of administration and will be apparent to those of skill in the art. A wide variety of suitable pharmaceutical compositions are described, for example, in Remington's Pharmaceutical Sciences, 20^{th} ed., 2001).

Formulations suitable for oral administration can consist of (a) liquid solutions, such as an effective amount of the active compound suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The PBI compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, subcutaneous administration and intravenous administration are the preferred methods of administration. A specific example of a suitable solution formulation may comprise from about 0.5-100 mg/ml compound and about 1000 mg/ml propylene glycol in water. Another specific example of a suitable solution formulation may comprise from about 0.5-100 mg/ml compound and from about 800-1000 mg/ml polyethylene glycol 400 (PEG 400) in water.

A specific example of a suitable suspension formulation may include from about 0.5-30 mg/ml compound and one or more excipients selected from the group consisting of: about 200 mg/ml ethanol, about 1000 mg/ml vegetable oil (e.g., corn oil), about 600-1000 mg/ml fruit juice (e.g., grapefruit juice), about 400-800 mg/ml milk, about 0.1 mg/ml carboxymethylcellulose (or microcrystalline cellulose), about 0.5 mg/ml benzyl alcohol (or a combination of benzyl alcohol and benzalkonium chloride) and about 40-50 mM buffer, pH 7 (e.g., phosphate buffer, acetate buffer or citrate buffer or, alternatively 5% dextrose may be used in place of the buffer) in water.

A specific example of a suitable liposome suspension formulation may comprise from about 0.5-30 mg/ml compound, about 100-200 mg/ml lecithin (or other phospholipid or mixture of phospholipids) and optionally about 5 mg/ml cholesterol in water. For subcutaneous administration of certain PBI compounds, a liposome suspension formulation including 5 mg/ml compound in water with 100 mg/ml lecithin and 5 mg/ml compound in water with 100 mg/ml lecithin and 5 mg/ml cholesterol provides good results.

The formulations of compounds can be presented in unit-dose or multi-dose sealed containers, such as ampules and vials. Injection solutions and suspensions can be prepared from sterile powders, granules, and tablets of the kind previously described.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents, discussed in more detail, below.

In therapeutic use for the treatment of HCV infection, the PBI compounds are administered to patients diagnosed with HCV infection at dosage levels suitable to achieve therapeutic benefit. By therapeutic benefit is meant that the administration of compound leads to a beneficial effect in the patient over time. For example, therapeutic benefit is achieved when the HCV titer or load in the patient is either reduced or stops increasing. Therapeutic benefit is also achieved if the administration of compound slows or halts altogether the onset of the organ damage or other adverse symptoms that typically accompany HCV infections, regardless of the HCV titer or load in the patient.

The PBI compounds and/or compositions thereof may also be administered prophylactically in patients who are at risk of developing HCV infection, or who have been exposed to HCV, to prevent the development of HCV infection. For example, the PBI compounds and/or compositions thereof may be administered to hospital workers accidentally stuck with needles while working with HCV patients to lower the risk of, or avoid altogether, developing an HCV infection.

Initial dosages suitable for administration to humans may be determined from in vitro assays or animal models. For example, an initial dosage may be formulated to achieve a serum concentration that includes the IC₅₀ of the particular PBI compound being administered, as measured in an in vitro assay. Alternatively, an initial dosage for humans may be based upon dosages found to be effective in animal models of HCV infection. Exemplary suitable model systems are described, for example, in Muchmore, 2001, Immunol. Rev. 183:86-93 and Lanford & Bigger, 2002, Virology, 293:1-9, and the referenced cited therein. As one example, the initial dosage may be in the range of about 0.01 mg/kg/day to about 200 mg/kg/day, or about 0.1 mg/kg/day to about 100 mg/kg/day, or about 1 mg/kg/day to about 50 mg/kg/day, or about 10 mg/kg/day to about 50 mg/kg/day, can also be used. The dosages, however, may be varied depending upon the requirements of the patient, the severity of the condition being treated, and the compound being employed. The size of the dose also will be determined by the existence, nature, and extent of any adverse side-effects that accompany the administration of a particular compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired or indicated.

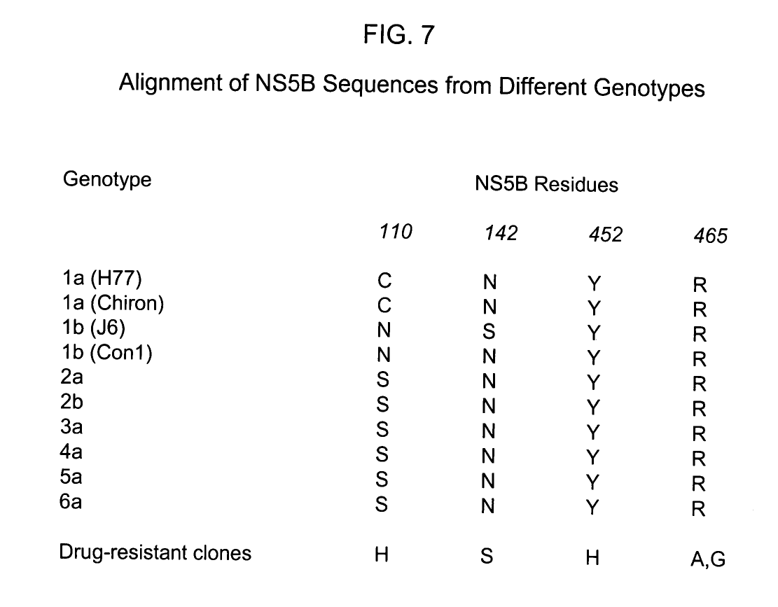

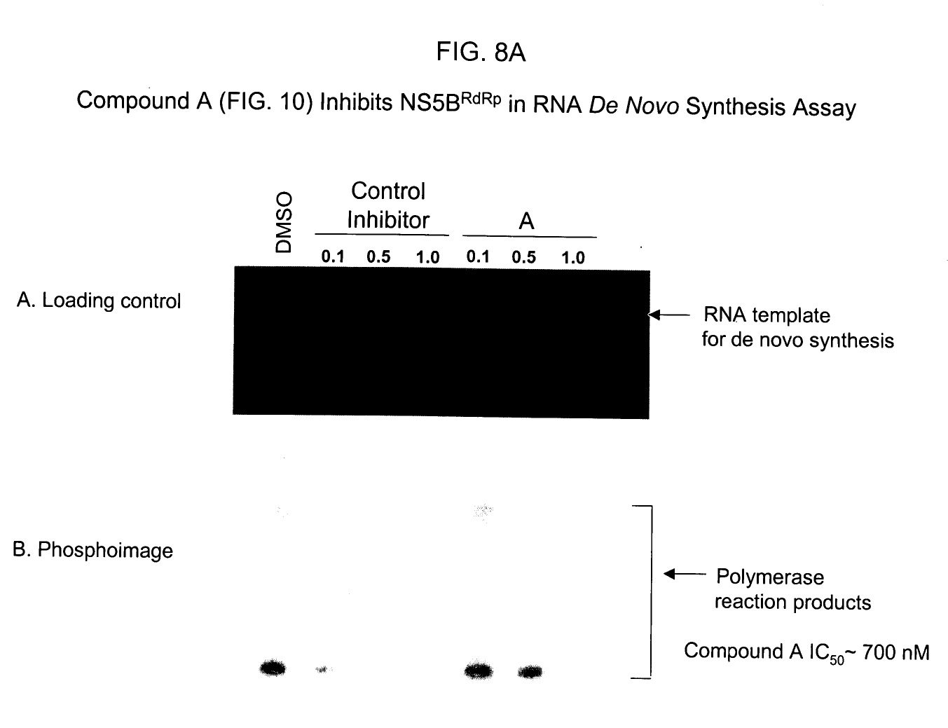

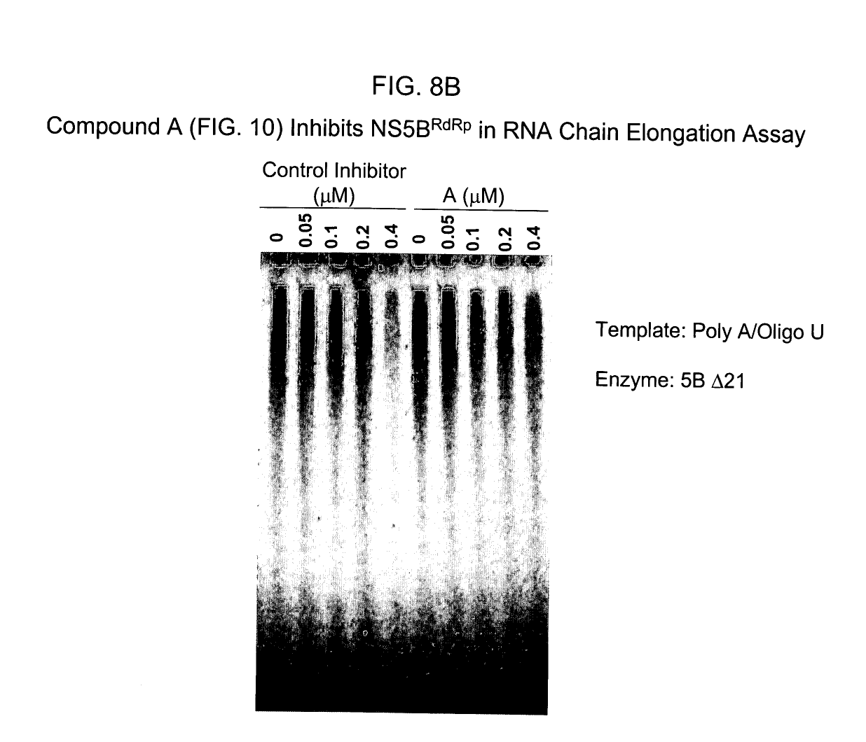

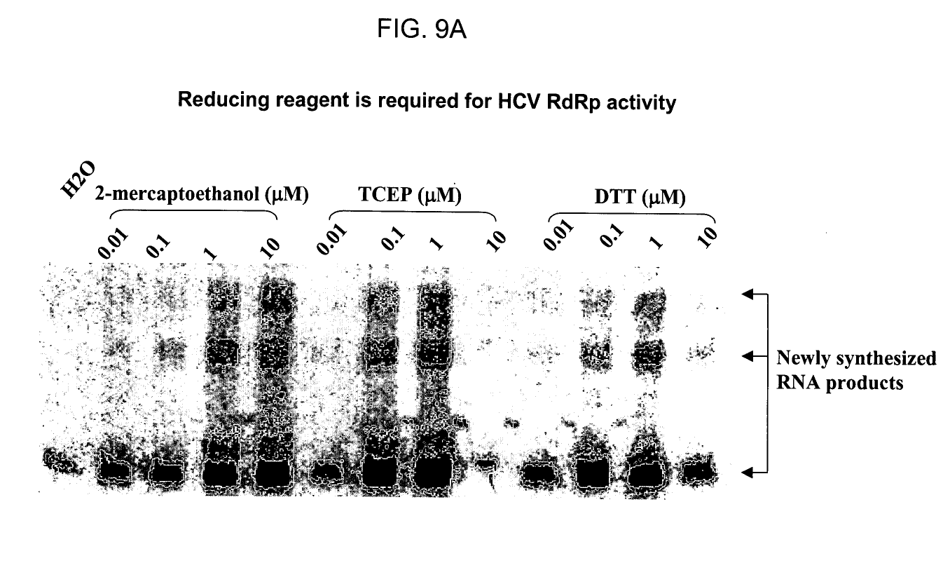

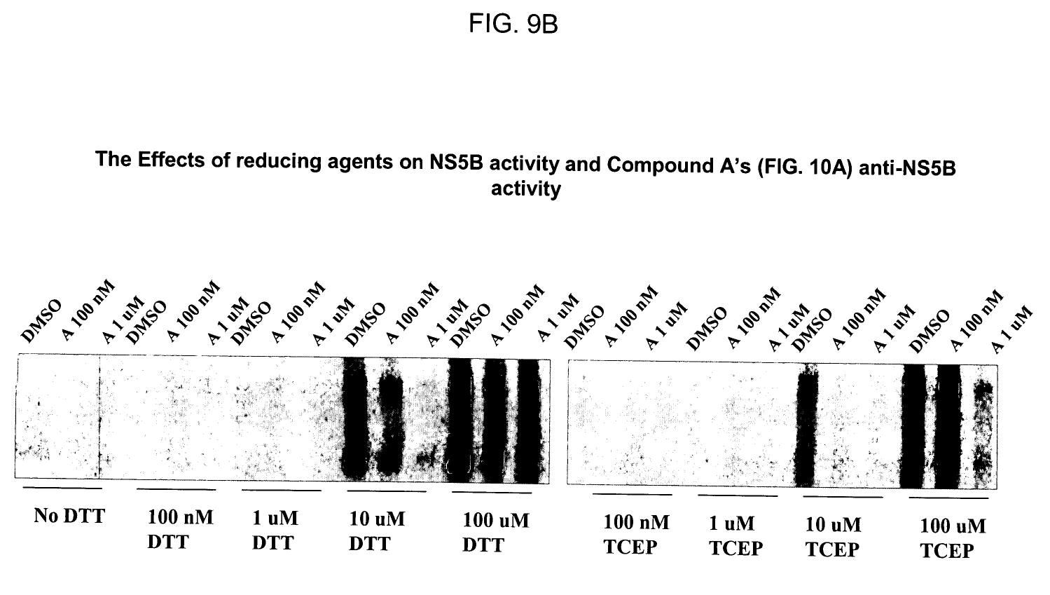

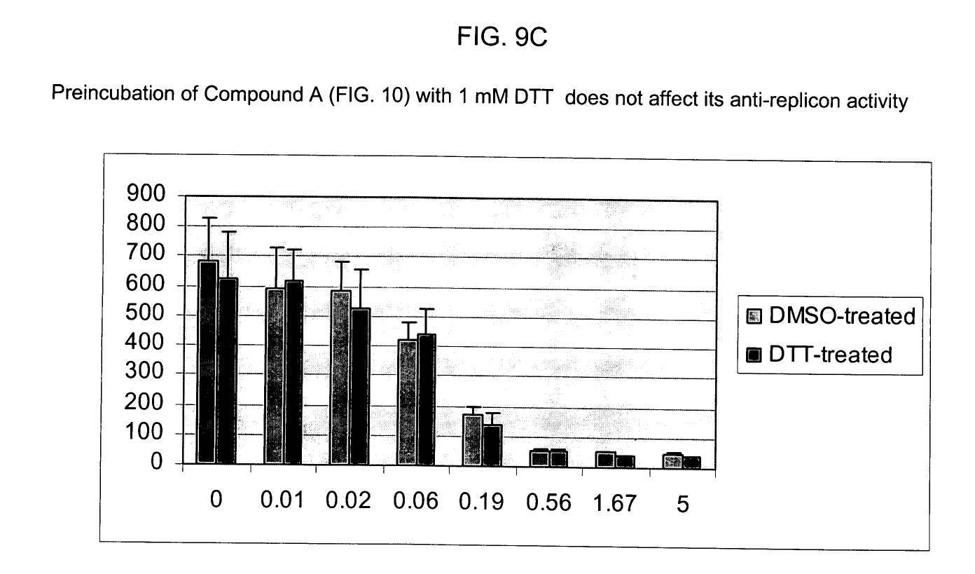

As mentioned previously, certain PBI compounds induced mutations in HCV NS5B polymerase residues that are highly conserved across all known HCV genotypes (see, e.g., FIGS. 2 and 7B). As a consequence, PBI compounds are expected to be useful as broad spectrum anti-HCV agents, being useful in the treatment or prophylaxis of HCV infections caused by any one of the HCV strains delineated in FIG. 7B, or combinations of such strains. As will be recognized by skilled artisans, such broad spectrum activity makes the PBI compounds ideally suited for use in combination with other HCV treatments that are known to be effective against only one or a few HCV genotypes. Importantly, the PBI compounds may be administered in situations where other known HCV treatments fail or in situations where patients develop resistance to, or fail to respond to, chronic treatment with other HCV treatments.

6.4 Combination Therapy

In certain embodiments, the PBI compounds and/or compositions thereof can be used in combination therapy with at least one other therapeutic agent. A PBI compound and/or composition thereof and the therapeutic agent can act additively or, more preferably, synergistically. The PBI compound and/or a composition thereof may be administered concurrently with the administration of the other therapeutic agent(s), or it may be administered prior to or subsequent to administration of the other therapeutic agent(s).

In some embodiments, the PBI compounds and/or compositions thereof are used in combination therapy with other antiviral agents or other therapies known to be effective in the treatment or prevention of HCV.

In a specific embodiment, combinations of agents known to inhibit HCV through different mechanisms and/or by binding to different proteins, or to different locations on the NS5B polymerase, are used. For example, as outlined in Wang et al., 2003, J. Biol. Chem. 278:9489, phenylalanaine derivative inhibitors have been shown to bind to the “thumb” region of the NS5B polymerase. Similarly, as the binding site for the NTPs appears to be yet another location on the molecule, nucleoside inhibitors, particularly ribonucleoside inhibitors (for example 3TC®), can be used in combination therapies. Similarly, other known NS5B inhibitors, including, but not limited to, rhodanines, barbituric acid derivatives, dihydroxypyrimidine carboxylic acids, dikeotacid derivatives, 2-methylidenylbenzothiophene compounds and pyrrolidine and benzimidazole analogs (see Wang et al. for the appropriate references, which are hereby incorporated by reference).

Additionally, the PBI compounds and/or compositions thereof may be used in combination with drugs that inhibit or act on different proteins or mechanisms, including for example known antivirals, such as ribavirin (see, e.g., U.S. Pat. No. 4,530,901). As another specific example, the PBI compounds and/or compositions thereof may also be administered in combination with one or more of the compounds described in any of the following: U.S. Pat. Nos. 6,143,715; 6,323,180; 6,329,379; 6,329,417; 6,410,531; 6,420,380; and 6,448,281, the disclosures of which are incorporated herein by reference.

In yet as another specific example, the PBI compounds and/or compositions thereof may be used in combination with interferons such as α-interferon, β-interferon and/or γ-interferon. The interferons may be unmodified, or may be modified with moieties such as polyethylene glycol (pegylated interferons). Many suitable unpegylated and pegylated interferons are available commercially, and include, by way of example and not limitation, recombinant interferon alpha-2b such as Intron-A interferon available from Schering Corporation, Kenilworth, N.J., recombinant interferon alpha-2a such as Roferon interferon available from Hoffmann-LaRoche, Nutley, N.J., recombinant interferon alpha-2C such as Berofor alpha 2 interferon available from Boehringer Ingelheim Pharmaceutical, Inc., Ridgefield, Conn., interferon alpha-n1, a purified blend of natural alpha interferons such as Sumiferon available from Sumitomo, Japan or as Wellferon interferon alpha-n1 (INS) available from the Glaxo-Wellcome Ltd., London, Great Britain, or a consensus alpha interferon such as those described in U.S. Pat. Nos. 4,897,471 and 4,695,623 (especially Examples 7, 8 or 9 thereof) and the specific product available from Amgen, Inc., Newbury Park, Calif., or interferon alpha-n3 a mixture of natural alpha interferons made by Interferon Sciences and available from the Purdue Frederick Colo., Norwalk, Conn., under the Alferon Tradename, pegylated interferon-2b available from Schering Corporation, Kenilworth, N.J. under the tradename PEG-Intron A and pegylated interferon-2a available from Hoffrnan-LaRoche, Nutley, N.J. under the tradename Pegasys.

As yet another specific example, the PBI compounds and/or compositions thereof may be administered in combination with both ribovirin and an interferon. As yet another specific example, the PBI compounds and/or compositions thereof may be administered in combination with HCV IRES inhibitors, such as those described in application Ser. No. 10/122,675, filed Apr. 12, 2002, which is incorporated herein by reference.

6.5 Assays

The present disclosure provides methods of identifying bioactive agents which bind the pocket region and/or modulate the activity of an HCV NS5B polymerase. Thus, the present disclosure provide assays for identifying additional PBI compounds. These assays include biochemical assays, using the known biochemical properties of NS5B and standard assay techniques, and in silico assays, using the crystallography information of the NS5B polymerase for use in docking experiments, de novo bioactive agent design, structure-activity relationship (SAR) modeling, etc.

6.5.1 Biochemical Assays

In one embodiment, the assay is a biochemical assay that generally comprises contacting an NS5B polymerase with a candidate agent and determining whether the candidate agent binds the pocket region of the NS5B polymerase.

6.5.1.1 General Reagents

The assay may employ a full-length NS5B polymerase, or a derivative, fragment, etc. as discussed above and all of which fall into the definition of an NS5B polymerase. The polymerase is preferably produced recombinantly, using well known techniques in the art; see Ago et al., supra; Lesburg et al., supra; Bressanelli et al., supra; and Wang et al., supra, all of which are incorporated herein for the techniques used to produce recombinant NS5B. Note that in general, the highly hydrophobic C-terminus is generally removed (usually roughly 21 residues) for expression purposes and that the addition of a hexahistidine to the N-terminus is well tolerated (and thus can facilitate the attachment of the protein to a solid support for use in heterogeneous assays as outlined below). Of particular interest are screening assays for PBIs that have a low toxicity for human cells. Thus, once identified, toxicity screens may be carried out as well, using well known techniques.

Screens may be designed to first find candidate agents that can bind to NS5B polymerases, and then these agents may be used in assays that evaluate the ability of the candidate agent to modulate NS5B activity. Thus, as will be appreciated by those in the art, there are a number of different assays which may be run; binding assays and activity assays.

In general, the biochemical assays are run under conditions known in the art. A variety of reagents in addition to the required reagents may be included in the screening assays. These include reagents like salts, neutral proteins, e.g. albumin, detergents, etc which may be used to facilitate optimal inhibitor-protein binding and/or reduce non-specific or background interactions. Also reagents that otherwise improve the efficiency of the assay, such as protease inhibitors, nuclease inhibitors (which may, anti-microbial agents, etc., may be used. The mixture of components may be added in any order that provides for the requisite binding. Generally a plurality of assay mixtures are run in parallel with different agent concentrations to obtain a differential response to the various concentrations (see for example the dilution series examples). Typically, one of these concentrations serves as a negative control, i.e., at zero concentration or below the level of detection. Some embodiments employ high throughput screening methods, which include the use of libraries of candidate agents, in formats that are useful for the use of robotic systems and rapid screening such as FACS, etc.

In some embodiments, the devices used in the methods described herein comprise liquid handling components, including components for loading and unloading fluids at each station or sets of stations. The liquid handling systems can include robotic systems comprising any number of components. In addition, any or all of the steps outlined herein may be automated; thus, for example, the systems may be completely or partially automated.

As will be appreciated by those in the art, there are a wide variety of components which can be used, including, but not limited to, one or more robotic arms; plate handlers for the positioning of microplates; holders with cartridges and/or caps; automated lid or cap handlers to remove and replace lids for wells on non-cross contamination plates; tip assemblies for sample distribution with disposable tips; washable tip assemblies for sample distribution; 96 well loading blocks; cooled reagent racks; microtitler plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In one embodiment, chemically derivatized particles, plates, cartridges, tubes, magnetic particles, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful in this invention.

In another embodiment, platforms for multi-well plates, multi-tubes, holders, cartridges, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, and multi-position work decks for source samples, sample and reagent dilution, assay plates, sample and reagent reservoirs, pipette tips, and an active wash station.

In another embodiment, thermocycler and thermoregulating systems are used for stabilizing the temperature of the heat exchangers such as controlled blocks or platforms to provide accurate temperature control of incubating samples from 4C to 100C; this is in addition to or in place of the station thermocontrollers.

In another embodiment, interchangeable pipet heads (single or multi-channel) with single or multiple magnetic probes, affinity probes, or pipetters robotically manipulate the components of the invention. Multi-well or multi-tube magnetic separators or platforms manipulate the components in single or multiple sample formats.

These instruments can fit in a sterile laminar flow or fume hood, or are enclosed, self-contained systems, for example for hazardous operations.

Flow cytometry or capillary electrophoresis formats can be used for individual capture of magnetic and other beads, as is generally more fully described below.

The flexible hardware and software allow instrument adaptability for multiple applications. The software program modules allow creation, modification, and running of methods. The system diagnostic modules allow instrument alignment, correct connections, and motor operations. The customized tools, labware, liquid, and/or particle transfer patterns allow different applications to be performed. The database allows method and parameter storage. Robotic and computer interfaces allow communication between instruments.

In one embodiment, the robotic apparatus includes a central processing unit that communicates with a memory and a set of input/output devices (e.g., keyboard, mouse, monitor, printer, etc.) through a bus. Again, as outlined below, this may be in addition to or in place of the CPU for the multiplexing devices. The general interaction between a central processing unit, a memory, input/output devices, and a bus is known in the art. Thus, a variety of different procedures, depending on the experiments to be run, are stored in the CPU memory. As is described below, in silico methods also rely on CPUs, which may be the same or different as those described for robotic systems.

6.5.2 Binding Assays

In one embodiment, the methods comprise contacting an NS5B polymerase with a candidate bioactive agent and determining whether the candidate agent binds to the pocket region of the NS5B polymerase. In some embodiments, as outlined herein, variant or derivative NS5B polymerases may be used, including deletion NS5B polymerases as outlined above. As will be appreciated by those in the art, there are a wide variety of possible assays to determine such binding (and/or modulation of activity), including both homogeneous and heterogeneous assay systems.

6.5.3 Heterogeneous Systems

Generally, heterogeneous systems are those which utilize both an aqueous phase and a solid support, to facilitate washing, etc. Accordingly, in one embodiment of the methods herein, the NS5B polymerase or the candidate agent is non-diffusably bound to an insoluble solid support having isolated sample receiving areas (e.g. a microtiter plate, an array, etc.), or beads. As defined above, the insoluble supports may be made of any composition to which the compositions can be bound, is readily separated from soluble material, and is otherwise compatible with the overall method of screening. The surface of such supports may be solid or porous and of any convenient shape. Examples of suitable insoluble supports include microtiter plates, arrays, membranes and beads. Microtiter plates and beads are especially convenient because a large number of assays can be carried out simultaneously, using small amounts of reagents and samples. The particular manner of binding of the composition is not crucial so long as it is compatible with the reagents and overall methods described herein, maintains the activity of the composition and is nondiffusable.

Specific methods of binding include, but are not limited to, the use of antibodies (which do not sterically block the pocket region when the protein is bound to the support), direct binding to “sticky” or ionic supports, chemical crosslinking, the synthesis of the protein or agent on the surface, as well as the use of fusion proteins when the NS5B is attached to the surface. For example, the use of epitope tags or His6 tags, particularly at the N-terminus or at the C-terminus (or at the C-terminus after the removal of the hydrophobic residues), allow the attachment of the fusion protein to the support. The attachment of the candidate agent will generally be done as is known in the art, and will depend on the composition of the candidate agent and the support. In general, the candidate agents are attached to the support through the use of functional groups on each that can then be used for attachment. Preferred functional groups for attachment are amino groups, carboxy groups, oxo groups and thiol groups. These functional groups can then be attached, either directly or through the use of a linker. Linkers are known in the art; for example, homo-or hetero-bifunctional linkers as are well known (see 1994 Pierce Chemical Company catalog, technical section on cross-linkers, pages 155-200, incorporated herein by reference, as well as similar chapters in more recent versions of the catalog). Specific suitable linkers include, but are not limited to, alkyl groups (including substituted alkyl groups and alkyl groups containing heteroatom moieties), with short alkyl groups, esters, amide, amine, epoxy groups and ethylene glycol and derivatives being preferred.

Following binding of the polymerase or agent, excess unbound material is preferably removed by washing. The sample receiving areas may then be blocked through incubation with bovine serum albumin (BSA), casein or other innocuous protein or other moieties if needed.

In one embodiment, the NS5B polymerase is bound to the support, and a candidate bioactive agent is added to the assay. Alternatively, the candidate agent is bound to the support and the NS5B polymerase is added.

Binding can be tested in a variety of ways. In one embodiment, a first binding test is done to determine if the candidate agents binds the NS5B polymerase generally, and then a competitive binding experiment with a known PBI is done to determine if the agent is bound to the pocket region. Alternatively, the competitive binding experiment may be carried out without first assaying for general binding.

In a specific embodiment, when the candidate agent has an aromatic group, binding can be tested by intrinsic fluorescence quenching of the fluorescent emission of the NS5B polymerase as detailed in the example section and shown in the FIGS.

In another specific embodiment, the non-bound component is labeled with a suitable label, as defined herein, and the support is washed after a suitable incubation time and then tested for the presence of the label. Additional embodiments utilize both components being labeled.

In another embodiment, fluorescence resonance energy transfer (FRET) testing is done. In this embodiment, both components comprise a different FRET label, as described above, such that upon binding, a FRET reaction occurs and can be detected.

In one specific embodiment, the polymerase is bound to a bead with a first label and the candidate agent is bound to a bead with a second label, with fluorescent labels being preferred. Upon binding of the agent to the polymerase, the two beads are associated. Two color FACS analysis can then be done for the detection of aggregates with both colors present.

6.5.4 Homogeneous Systems

Homogeneous assays are typically carried out in solution without immobilizing the assay reagents on solid supports. Myriad different homogeneous binding assays, and in particular competitive binding assays, are well-known, as are methods for carrying them out. Any of these assays may be used to screen for or identify PBI compounds.

6.5.5 Testing for Pocket Binding

In general, there are several methods that can be used to ascertain whether the candidate agent is bound in the pocket region. One embodiment utilizes competitive assays, wherein known PBIs are used to either displace or be displaced by the candidate agent. Such assays can be carried out in solution or with the aid of support-immobilized NS5B polymerase or candidate agent, as is known in the art.

In a specific embodiment of such a competitive assay, particularly in the case where higher affinity agents are desired, the assay is done using a labeled known PBI, such as those depicted in FIG. 10 or described in the referenced applications. That is, the NS5B polymerase is generally bound to the support, a labeled PBI is added, the support is washed to eliminate non-specific binding and then the candidate agent is added. In some embodiments, the polymerase may have the bound PBI prior to the attachment reaction (this may be beneficial for quantifying protein binding to the support). Similarly, in some instances, the candidate agent may be differentially labeled. Thus, when the candidate agent is unlabeled, a loss of support-associated label is an indication of pocket region binding, and generally will indicate a higher affinity, depending on equilibria phenomenon, as discussed below. If the candidate agent is labeled, a simultaneous loss of the PBI signal and a corresponding increase in the candidate agent signal thus identifies the agent as an additional PBI.

In an additional embodiment, the known PBI is bound to the support, the NS5B polymerase added to form a polymerase-PBI complex, and excess polymerase washed off. Aliquots of candidate agents are added, incubated for a suitable time period, and then the supernatant is tested for the presence of the polymerase, indicating competitive release of the polymerase. In this embodiment, the polymerase is preferably labeled, to allow detection of initial binding to the PBI, as well as track the loss of signal, and thus of competitive binding, from the support. However, other methods to quantify the amount of polymerase both on the support and released from it are also known, for example immunoassays may be done. Again, this label is an indication of pocket region binding, and generally will indicate a higher affinity, depending on equilibria phenomenon.

In a further embodiment, the candidate agents are bound to a support (or supports, such as beads). As for all the assays herein, this can be done individually or in pools, if large numbers of candidate agents are to be tested; any pool that tests “positive”, e.g. shows pocket domain binding and/or inhibitory activity, may be broken down with each agent in the pool being retested.

When the candidate agents are bound to the support, aliquots of the NS5B polymerase are added, allowed to incubate for a suitable time period, rinsed, and then the known PBI is added. This can allow the discovery of agents with lower affinity than the known PBI, again depending on equilibria. Again, the polymerase is preferably labeled, to allow detection of initial binding to the candidate agent, as well as track the loss of signal, and thus of competitive binding, from the support. However, other methods to quantify the amount of polymerase both on the support and released from it are also known; for example immunoassays may be done. Furthermore, the known PBI may be differentially labeled, to allow the tracking of the competitive binding. In some cases, the polymerase may be attached to a labeled bead, and then the effluent from the competition assay may be tested by FACS for the presence of both the bead label and the known PBI label.

Competitive binding assays in these formats may also be run as FRET assays in a variety of ways. The NS5B polymerase can comprise a first FRET label, and either the known PBI or candidate agent comprises a second FRET label. Depending on the format, either a gain in a FRET signal (e.g. the candidate agent has the FRET label and the PBI does not) or a loss (vice versa) can be an indication of candidate agent binding to the exclusion of the PBI.

Any of the assays described herein may include a preferably labeled non-pocket binding inhibitor, such as a TSI. This adds additional confidence that the candidate agent is binding to a different location than the TSI. Thus, the presence of the TSI label is checked as well, as it should be present at all times. In addition, assays using TSIs may be done as FRET assays. For example, a TSI labeled with a first FRET label and either a known PBI or a candidate agent with a second FRET label can be used. Depending on the format, either a gain in a FRET signal (e.g. the candidate agent has the FRET label and the PBI does not) or a loss (vice versa) can be an indication of candidate agent binding. FRET assays may also be done in homogeneous systems described below, as “washing” is not required in some instances.

Any or all of these experiments can be subjected to altered experimental conditions and retested. This may be done, for example, to quantify or alter the binding affinity of the candidate agent for the target. Thus, for example, changes in pH, temperature, buffer, salt concentration, the identity and/or concentration of reducing agent, etc. can be made. In a preferred embodiment, the pH is changed, generally by increasing or decreasing the pH, usually by from about 0.5 to about 3 pH units. Alternatively, the temperature is altered, with increases or decreases of from about 5° C. to about 30° C. being preferred. Similarly, the salt concentration may be modified, with increases or decreases of from about 0.1 M to about 2 M being preferred.

Candidate compounds may also be screened for binding the pocket region using NMR spectroscopy techniques that are well-known in the art. In one exemplary method, described in U.S. Pat. No. 5,698,401, the disclosure of which is incorporated herein by reference, a two-dimensional ¹H/¹⁵N correlation spectrum of an ¹⁵N-labeled target molecule is obtained. The labeled target is then exposed to a candidate compound and a second two-dimensional ¹H/¹⁵N correlation spectrum obtained. Comparison of the two correlation spectra reveals whether the candidate compound bound the target. As will be appreciated by skilled artisans, the method is ideally suited to identifying PBI compounds. Because the chemical shift values of the particular ¹H/¹⁵N peaks in the correlation spectra correspond to known specific locations of atomic groupings in the target molecule (e.g., the N-H atoms of an amide or peptide linkage of a particular amino acid residue in a polypeptide), the method permits not only the identification of candidate compounds that bind the NS5B polymerase, but also the identification of compounds that bind the NS5B polymerase at the Rigel pocket. The dissociation constant of an identified PBI compound can also be determined by this method. Depending upon the identity of the candidate compound screened, the NS5B polymerase or the candidate compound, or both can be labeled. In one embodiment, the NS5B polymerase is labeled and the candidate compound is unlabeled. A similar method that utilizes one-dimensional NMR spectroscopy that may be employed is described in U.S. Pat. No. 6,043,024, the disclosure of which is incorporated herein by reference.

As will be appreciated by those in the art, crystallizing the complex of the NS5B polymerase and the candidate agent and solving the structure is also a way to confirm binding in the pocket region.

6.5.6 Testing for Modulation of Activity

In some cases, preliminary binding assays may not be done; rather, activity assays can be run with the use of competition assays serving to ensure that the agents are binding to the pocket region.

The activity assays may investigate any parameter that is directly or indirectly under the influence of HCV, including, but not limited to, protein-RNA binding, translation, transcription, genome replication, protein processing, viral particle formation, infectivity, viral transduction, etc In particular, the NS5B polymerase has a number of suitable bioactivity assays that can be run to determine the inhibitory effect of the pocket binding candidate agent, including, but not limited to, nucleic acid synthesis assays, RNA binding assays, and oligomerization assays, both with other NS5B molecules as well as other HCV proteins.

The general NS5B activity assays are well known in the art. Specific examples of suitable assays are described in Wang et al., 2003, J. Biol. Chem. 278(11):9489-9495; Gosert et al., 2003, J. Virol. 77(9):5487-5492; Dimitrova et al., 2003, J. Virol. 77(9):5401-5414; Qin et al., 2002, J. Biol. Chem. 277(3):2132-2137; Wang et al., 2002, J. Virol. 76(8):3865-3872; Piccininni et al., 2002, J. Biol. Chem. 227(47):45670-45679; Shirota et al., 2002, J. Biol. Chem. 277(13):11149-11155; Hwang et al., 1997, Virology 6:439-446; and Ishido et al., 1997, J. Virol. 6:6465-6471.

In general, the assays are run in triplicate, with one sample serving as the positive control, one with a known PBI, and one with a candidate agent already shown to be a pocket binding moiety. Again, different components of the assays may be labeled as needed. In many cases, the activity assays are better run as homogeneous systems, e.g. in solution phase, to allow for optimum assay conditions. IC₅₀s, LD₅₀s, K_{D}S and K_{I}s for the candidate agent are all determined using known techniques as are well known in the art.

In one embodiment, assays are run with candidate agents using variant NS5B polymerases which are resistant to the general known class of PBI inhibitors (e.g. the strains with mutations at one or more of positions 110, 142, 148, 213, 316, 444, 445, 447, 451, 452 and/or 465, with strains with alterations at positions selected from the group consisting of 110, 142, 452 and 465 being particularly preferred, most preferably at position 465, as all variants discovered to date possess a variation at this site). That is, if a resistant strain is similarly resistant to the candidate agent being tested, this is a good indication that it is a PBI. These assays are generally run in triplicate as noted above, with different variants being tested with the candidate agent under question.

Once identified as a PBI, additional testing may be done, for example, to examine the extent of inhibition, samples, cells, tissues, etc. comprising an HCV replicon or HCV RNA are treated with the PBI and the value for the parameter compared to control cells (untreated or treated with a vehicle, other placebo or other known PBI such as structures A and C). Control samples are assigned a relative activity value of 100%. Inhibition is achieved when the activity value of the test compound relative to the control is about 90%, preferably 50%, and more preferably 25-0%.

Alternatively, the extent of inhibition may be determined based upon the IC₅₀ of the compound in the particular assay, as described herein. In one embodiment, the inhibitory activity of the compounds can be confirmed in a replicon assay that assesses the ability of a test compound to block or inhibit HCV replication in replicon cells. One example of a suitable replicon assay is the liver cell-line Huh 7-based replicon assay described in Lohmann et al., 1999, Science 285:110-113. Specific examples of this replicon assay which utilize luciferase translation are described in WO 03/040112 and WO 2004/018463, the disclosures of which are incorporated herein by reference. In one embodiment of this assay, the amount of test compound that yields a 50% reduction in translation as compared to a control cell (IC₅₀) may be determined.

Alternatively, the inhibitory activity of the compounds can be confirmed using a quantitative Western immunoblot assay utilizing antibodies specific for HCV non-structural proteins, such as NS3, NS4A NS5A and NS5B. In one embodiment of this assay, replicon cells are treated with varying concentrations of test compound to determine the concentration of test compound that yields a 50% reduction in the amount of a non-structural protein produced as compared to a control sample (IC₅₀). A single non-structural protein may be quantified or multiple non-structural proteins may be quantified. Antibodies suitable for carrying out such immunoblot assays are available commercially (e.g., from BIODESIGN International, Saco, Me.).

Alternatively, the inhibitory activity of the compounds may be confirmed in an HCV infection assay, such as the HCV infection assay described in Fournier et al., 1998, J. Gen. Virol. 79(10):2367:2374, the disclosure of which is incorporated herein by reference. In one embodiment of this assay, the amount of test compound that yields a 50% reduction in HCV replication or proliferation as compared to a control cell (IC₅₀) may be determined. The extent of HCV replication may be determined by quantifying the amount of HCV RNA present in HCV infected cells. A specific method for carrying out such an assay is provided in the Examples section.

As yet another example, the inhibitory activity of the compounds can be confirmed using an assay that quantifies the amount of HCV RNA transcribed in treated replicon cells using, for example, a Taqman assay (Roche Molecular, Alameda, Calif.). In one embodiment of this assay, the amount of test compound that yields a 50% reduction in transcription of one or more HCV RNAs as compared to a control sample (IC₅₀) may be determined.

It should also be noted that antibodies can be raised to the pocket region using known techniques and then used in competitive assays.

In addition, there are some activities associated with locations other than the pocket binding region of the NS5B polymerase that can be used as negative controls, such as the nucleotide binding site, the catalytic domain assays, etc.

6.5.7 In Silico Assays

As will be appreciated by those skilled in the art, there are a wide variety of in silico assays to determine pocket domain binding, and to perform candidate agent modeling and optimization. Generally speaking, the general approach is to use the structural coordinates of any HCV NS5B polymerase, including those cited herein, particularly the coordinates of the residues defining or comprising the pocket domain, to design, develop, optimize and/or analyze candidate agents to find bioactive agents, e.g. PBIs. A wide variety of available methodologies are described below, as well as in U.S. Pat. Nos. 5,856,116; 6,128,582; 6,153,579; 6,273,589; 6,343,257; and 6,387,641, all of which are incorporated herein by reference.

In one embodiment, PBIs may be identified by computationally screening small molecule databases for chemical entities or compounds that can bind in whole, or in part, to the pocket region of the NS5B polymerase. In this screening, the quality of fit of such entities or compounds to the binding site may be judged either by shape complementarity or by estimated interaction energy. Meng, et al., 1992, J. Comp. Chem. 13:505-524.

In addition, in accordance with this disclosure, all or part of the NS5B polypeptide (but definitely including the pocket region) may be crystallized in co-complex with known PBIs. The crystal structures of a series of such complexes may then be solved by molecular replacement and compared with that of the polymerase in the absence of the inhibitor. Inhibitor interaction sites are thus identified. This information provides an additional tool for determining the most efficient binding interactions, for example, increased hydrophobic interactions, electrostatic, van der Waals, hydrogen bonding, etc. between the protein and a candidate agent.

In addition, since the PBIs and the TSIs bind in different locations, crystals of complexes with both inhibitors may also find use in the design of useful inhibitors.

All of the complexes referred to above may be studied using well-known X-ray diffraction techniques and may be refined versus 2-3.Å resolution X-ray data to an R value of about 0.20 or less using computer software, such as X-PLOR (Yale University, © 1992, distributed by Molecular Simulations, Inc.). See, e.g., Blundel & Johnson, supra; Methods in Enzymology, vol. 114 & 115, H. W. Wyckoff et al., eds., Academic Press (1985). This information may thus be used to optimize known PBIs, and also to design and synthesize novel classes of PBIs.

The design of compounds that bind to or inhibit NS5B according to the principles described herein generally involves consideration of two factors. First, the compound should be capable of physically and structurally associating with the protein. Non-covalent molecular interactions important in the association of NS5B with suitable PBIs include hydrogen bonding, van der Waals, electrostatic interactions and hydrophobic interactions.

Second, the candidate agent should be able to assume a conformation that allows it to associate with the pocket region of the protein. Although certain portions of the agent will not directly participate in this association with the protein, those portions may still influence the overall conformation of the molecule. This, in turn, may have a significant impact on potency. Such conformational requirements can include the overall three-dimensional structure and orientation of the chemical entity or compound in relation to all or a portion of the pocket region binding site, or the spacing between functional groups of a candidate agent comprising several chemical entities that directly interact with the protein.

Thus, the potential inhibitory or binding effect of a candidate agent on NS5B may be analyzed prior to its actual synthesis and testing by the use of computer modeling techniques. If the theoretical structure of the given compound suggests insufficient interaction and association between it and the pocket region of the protein, synthesis and testing of the compound is obviated. However, if computer modeling indicates a strong interaction, the molecule may then be synthesized and tested for its ability to bind to the pocket region and inhibit activity using activity assays outlined herein.

A potential PBI may be computationally evaluated and designed by means of a series of steps in which chemical entities or fragments are screened and selected for their ability to associate with the pocket region.

One skilled in the art may use one of several methods to screen candidate agents (or fragments thereof) for their ability to associate with the pocket region of the NS5B polymerase. This process may begin by visual inspection of, for example, the pocket region on the computer screen based on the NS5B structural coordinates. Selected fragments or chemical entities may then be positioned in a variety of orientations, or docked, within the pocket region as defined supra. Docking may be accomplished using software such as Quanta and Sybyl, followed by energy minimization and molecular dynamics with standard molecular mechanics force fields, such as CHARMM and AMBER.

Specialized computer programs may also assist in the process of selecting fragments or chemical entities. These include:

1. GRID (Goodford, P. J., “A Computational Procedure for Determining Energetically Favorable Binding Sites on Biologically Important Macromolecules”, J. Med. Chem., 28, pp. 849-857 (1985)). GRID is available from Oxford University, Oxford, UK.

2. MCSS (Miranker, A. and M. Karplus, “Functionality Maps of Binding Sites: A Multiple Copy Simultaneous Search Method.” Proteins: Structure. Function and Genetics, 11, pp. 29-34 (1991)). MCSS is available from Molecular Simulations, Burlington, Mass.

3. AUTODOCK (Goodsell, D. S. and A. J. Olsen, “Automated Docking of Substrates to Proteins by Simulated Annealing”, Proteins: Structure. Function, and Genetics, 8, pp. 195-202 (1990)). AUTODOCK is available from Scripps Research Institute, La Jolla, Calif.

4. DOCK (Kuntz, I. D. et al., “A Geometric Approach to Macromolecule-Ligand Interactions”, J. Mol. Biol., 161, pp. 269-288 (1982)). DOCK is available from University of California, San Francisco, Calif.

Once suitable chemical entities or fragments have been selected, they can be assembled into a single compound or inhibitor. Assembly may be proceed by visual inspection of the relationship of the fragments to each other on the three-dimensional image displayed on a computer screen in relation to the structure coordinates of the pocket region of NS5B. This can be followed by manual model building using software such as Quanta or Sybyl.

Useful programs to aid one of skill in the art in connecting the individual chemical entities or fragments include:

1. CAVEAT (Bartlett, P. A. et al, “CAVEAT: A Program to Facilitate the Structure-Derived Design of Biologically Active Molecules”. In “Molecular Recognition in Chemical and Biological Problems”, Special Pub., Royal Chem. Soc., 78, pp. 182-196 (1989)). CAVEAT is available from the University of California, Berkeley, Calif.

2. 3D Database systems such as MACCS-3D (MDL Information Systems, San Leandro, Calif.). This area is reviewed in Martin, Y. C., “3D Database Searching in Drug Design”, J. Med. Chem., 35, pp. 2145-2154 (1992)).

3. HOOK (available from Molecular Simulations, Burlington, Mass.).

Instead of proceeding to build a potential PBI in a step-wise fashion one fragment or chemical entity at a time as described above, potential PBIs may be designed as a whole or “de novo” using either an empty pocket region or optionally including some portion(s) of a known inhibitor(s). These methods include:

1. LUDI (Bohm, H.-J., “The Computer Program LUDI: A New Method for the De Novo Design of Enzyme Inhibitors”, J. Comp. Aid. Molec. Design, 6, pp. 61-78 (1992)). LUDI is available from Biosym Technologies, San Diego, Calif.

2. LEGEND (Nishibata, Y. and A. Itai, Tetrahedron, 47, p. 8985 (1991)). LEGEND is available from Molecular Simulations, Burlington, Mass.

3. LeapFrog (available from Tripos Associates, St. Louis, Mo.).

4. The Molecular Similarity application of QUANTA (Molecular Simulations Inc., San Diego, Calif.) version 4.1. The Molecular Similarity application permits comparisons between different structures, different conformations of the same structure, and different parts of the same structure. The procedure used in Molecular Similarity to compare structures is divided into four steps: 1) load the structures to be compared; 2) define the atom equivalences in these structures; 3) perform a fitting operation; and 4) analyze the results.

5. Specific computer software is available in the art to evaluate compound deformation energy and electrostatic interactions. Examples of programs designed for such uses include: Gaussian 94, revision C (M. J. Frisch, Gaussian, Inc., Pittsburgh, Pa. © 1995); AMBER, version 4.1 (P. A. Kollman, University of California at San Francisco, .©. 1995); QUANTA/CHARMM (Molecular Simulations, Inc., San Diego, Calif. © 1995); Insight II/Discover (Molecular Simulations, Inc., San Diego, Calif. © 1995); DelPhi (Molecular Simulations, Inc., San Diego, Calif. © 1995); and AMSOL (Quantum Chemistry Program Exchange, Indiana University). These programs may be implemented, for instance, using a Silicon Graphics workstation such as an Indigo.sup.2 with “IMPACT” graphics. Other hardware systems and software packages will be known to those skilled in the art.

Each structure is identified by a name. One structure is identified as the target (i.e., the fixed structure, which in this case would be the pocket region of the protein); all remaining structures are working structures (i.e., moving structures). Since atom equivalency within QUANTA is defined by user input, for the purpose of this embodiment, equivalent atoms are defined as protein backbone atoms (N, Ca, C and O) for all conserved residues between the two structures being compared. In addition, rigid fitting operations are preferred.

When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atom is an absolute minimum. This number, given in angstroms, is reported by QUANTA.

For the purpose of this disclosure, any molecule or molecular complex or binding pocket thereof that has a root mean square deviation of conserved residue backbone atoms (N, Ca, C, O) of less than 1.5 Å when superimposed on the relevant backbone atoms described by structure coordinates are considered identical. More preferably, the root mean square deviation is less than 1.0 Å.

The term “root mean square deviation” means the square root of the arithmetic mean of the squares of the deviations from the mean. It is a way to express the deviation or variation from a trend or object.

Once a potential PBI has been designed or selected by the above methods, the efficiency with which that potential PBI may bind to the pocket region of NS5B may be tested and optimized by computational evaluation. For example, an effective PBI must preferably demonstrate a relatively small difference in energy between its bound and free states (i.e., a small deformation energy of binding). Thus, the most efficient PBIs should preferably be designed with a deformation energy of binding of not greater than about 10 kcal/mole, preferably, not greater than 7 kcal/mole. Potential PBIs may interact with the polymerase in more than one conformation that is similar in overall binding energy. In those cases, the deformation energy of binding is taken to be the difference between the energy of the free compound and the average energy of the conformations observed when the inhibitor binds to the protein.

A potential PBI may be further computationally optimized so that in its bound state it would preferably lack repulsive electrostatic interaction with the polymerase. Such non-complementary (e.g., electrostatic) interactions include repulsive charge-charge, dipole-dipole and charge-dipole interactions. Specifically, the sum of all electrostatic interactions between the inhibitor and the polymerase when the complex is formed preferably make a neutral or favorable contribution to the enthalpy of binding.

Specific computer software is available in the art to evaluate compound deformation energy and electrostatic interaction. Examples of programs designed for such uses include: Gaussian 92, revision C>M. J. Frisch, Gaussian, Inc., Pittsburgh, Pa. © 1992; AMBER, version 4.0>P. A. Kollman, University of California at San Francisco, © 1994; QUANTA/CHARMM >Molecular Simulations, Inc., Burlington, Mass. © 1994; and Insight II/Discover (Biosysm Technologies Inc., San Diego, Calif. 1994). These programs may be implemented, for instance, using a Silicon Graphics workstation, IRIS 4D/35 or IBM RISC/6000 workstation model 550. Other hardware systems and software packages will be known to those skilled in the art.

Once a potential PBI has been optimally selected or designed, as described above, substitutions may then be made in some of its atoms or side groups in order to improve or modify its binding properties. This may also be done in conjunction with physically synthesizing the molecule and testing it biochemically. Generally, initial substitutions are conservative, i.e., the replacement group will have approximately the same size, shape, hydrophobicity and charge as the original group. It should, of course, be understood that components known in the art to alter conformation should be avoided. Such substituted chemical compounds may then be analyzed for efficiency of fit by the same computer methods described in detail, above.

Generally, once promising candidates are discovered in silico, they are synthesized and tested in any of the biochemical binding and/or activity assays described herein.

As will be recognized by skilled artisans, all of the various in silico methods described herein may utilize the atomic structure coordinates of the full-length NS5B polymerase, or subsets thereof that include only those residues defining the pocket. In one embodiment, the in silico methods utilize the atomic structure coordinates of only residues 440-470 of the NS5B polymerase (using the numbering scheme of Bressanelli et al., supra). Any of the known sets of structure coordinates may be used, including, for example, the structure coordinates found at the Protein Data Bank under deposit nos. 1CSJ, 1C2P or 1QUV, and in U.S. Pat. No. 6,434,489, the disclosures and coordinates of which are incorporated herein by reference. Alternatively, new sets may be obtained compirically by crystallizing the NS5B, preferably in co-complex with a known PBI, such as one of the PBIs of FIG. 10, as is described above.

The various docking and/or design techniques may be applied to identify and/or design compounds that contact, associate with and/or interact with specified residues of the NS5B polymerase. Suitable residues include those previously described. In one embodiment, candidate agents are screened or designed to interact with the side-chain of the residue at position 465 (typically an Arg residue in wild-type NS5B polymerase) and/or the side-chain of the residue at position 452 (typically a Tyr residue in wild-type NS5B polymerases) and optionally the side chains of one or more of the residues at the following positions: 142, 148, 213, 316, 444, 445, 447 and 451. In another embodiment, candidate agents are selected or designed to interact with the side chains of any residues included in the beta strands designated “17” or “18” and/or the alpha helix designated “R” in instant FIG. 12 and FIG. 2 of Bresanelli et al., supra.

PBI compounds may also be designed using NMR spectroscopic techniques, such as the technique described in U.S. Pat. No. 5,891,643, the disclosure of which is incorporated herein by reference.

6.6 The PBI Compounds

As discussed above, the disclosure provides assays and methods utilizing PBIs. Any PBI may be used in the various methods and assays described herein. Specific known PBIs, as well as the methods for their synthesis, are described above and in detail in the referenced applications. In addition, any of the PBI compounds identified by the methods described herein or other methods may be utilized in the various methods and assays.

### 7. EXAMPLES

The following examples are provided by way of illustration only and not by way of limitation. Those of skill in the art will readily recognize a variety of noncritical parameters that could be changed or modified to yield essentially similar results.

7.1.1 Replicon Assay and Counterscreens

The elucidation of the mechanism of action for the specific PBI compounds illustrated in FIG. 10 was based on counter screens of an HCV replicon assay.

The original replicon assay was as follows. The HCV replicon can include such features as the HCV 5′ untranslated region including the HCV IRES, the HCV 3′ untranslated region, selected HCV genes encoding HCV polypeptides, selectable markers, and a reporter gene such as luciferase, GFP, etc. In the assay, actively dividing 5-2 Luc replicon-comprising cells (obtained from Ralf Bartenschlager; see Lohmann et al., 1999, Science 285:110-113) were seeded at a density of between about 5,000 and 7,500 cells/well onto 96 well plates (about 90 μl of cells per well) and incubated at 37° C. and 5% CO₂ for 24 hours. Then, the test compound (in a volume of about 10 μl) was added to the wells at various concentrations and the cells were incubated for an additional 24 hours before luciferase assay. Briefly, the Bright-Glo reagent was diluted 1:1 with PBS and 100 μl of diluted reagent was added to each well. After 5 min of incubation at room temperature, luciferin emission was quantified with a luminometer. In this assay, the amount of test compound that yielded a 50% reduction in luciferase activity (IC₅₀) was determined.

Compounds identified in these original screens, particularly the structures depicted in FIG. 10A-C, were re-run in counterscreens to identify resistant replicons. A number of such replicons were isolated and cloned, and the clones again run against the compounds, confirming resistance; see the Figures. A chart showing NS5B mutations is provided in FIG. 2.

SAR testing was done, resulting in a large number of compounds exhibiting inhibition of at least one of the biochemical activities of the HCV NS5B polymerase, the structures of which are shown in the appendices attached hereto.

In addition, these compounds show high selectivity to HCV, while inactive in other viral replication systems, such as those of bone viral diarrhea virus (BVDV), yellow fever virus (YFV), poliovirus (PV), and GBV-B virus.

7.1.2 Replication Requires Reducing Agent

The assays utilized herein should generally include reducing agents. As discovered herein, the activity of the HCV NS5B polymerase requires the presence of reducing agents such as but not limited to, dithiothreitol (DTT), β-mercaptol ethanol (β-ME), and Tri(2-carboxyethyl) phosphine hydrochloride (TCEP). Moreover, Structure C of FIG. 10 and its analogues bind and inhibit only the active form of the NS5B polymerase, and the inhibition will be attenuated by excessive amount of reducing agent in the assay system (see Figures). DTT and other reducing agents do not chemically react with Structure C or the analogues, nor do they reduce the activity of this class of inhibitors in the cell based replicon assay (figures). Accordingly, the assays should be run with an optimal amount of reducing agent as a tool to screen for inhibitors bearing similar properties as Structure C and others outlined herein. Simple assays using a variety of reducing agent concentrations can be done to find the optimal concentration as is known in the art.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

1. A method of inhibiting an HCV NS5B polymerase, comprising the step of contacting the polymerase with a PBI compound.

2. The method of claim 1 in which the PBI compound contacts, associates with and/or interacts with a region of the NS5B polymerase selected from:
an NS5B polymerase residue selected from positions 142, 148, 213, 316, 444, 445, 447, 451, 452, 465 and combinations thereof;
an NS5B polymerase residue positioned within alpha helix “O”, alpha helix “P”, alpha helix “R”, beta strand “17” and beta strand “18” as defined in FIG. 12;
an NS5B polymerase residue positioned within beta strand “17” and alpha helix “P”, “O” and/or “R” as defined in FIG. 12;
an NS5B polymerase residue positioned within beta strand “17” and alpha helix “P”, “O” and/or “R” as defined in FIG. 12; and
an NS5B polymerase residue positioned in a region defined by residues 440 to 470.

3. The method of claim 1 in which the PBI compound comprises the following structure: 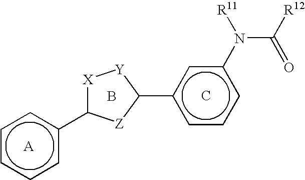
wherein:
the “A” ring is a substituted phenyl or pyridyl;
the “B” ring is saturated, unsaturated or aromatic and includes one or more heteroatoms at positions X, Y and Z which are selected from NH, N, O and S, with the proviso that X and Y are not both simultaneously O;
the “C” ring comprises a phenyl or pyridyl which may optionally include additional unillustrated substituents;
R¹¹ is hydrogen or alkyl; and
R¹² is mono- or di-halo methyl.

4. The method of claim 3 in which the PBI compound has one or more features selected from the group consisting of:
the “A” or “C” ring is a pyridyl;
the “A” and “C” rings are each phenyl; and
the “A” and “C” rings are not both phenyl.

5. The method of claim 4 in which the PBI compound is selected from structures A, B, C and D of FIG. 10.

6. The method of claim 1 in which the PBI compound is selected form the group consisting of an antibody or binding fragment thereof, a nucleic acid and an RNA.

7. The method of claim 1 in which the PBI compound competes for binding the NS5B polymerase with a second PBI compound comprising the structure: 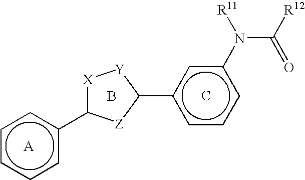
wherein:
the “A” ring is a substituted phenyl or pyridyl;
the “B” ring is saturated, unsaturated or aromatic and includes one or more heteroatoms at positions X, Y and Z which are selected from NH, N, O and S, with the proviso that X and Y are not both simultaneously O;
the “C” ring comprises a phenyl or pyridyl which may optionally include additional unillustrated substituents;
R¹¹ is hydrogen or alkyl; and
R¹² is mono- or di-halo methyl.

8. The method of claim 7 in which the second PBI compound has one or more features selected from the group consisting of:
the “A” or “C” ring is a pyridyl;
the “A” and “C” rings are each phenyl; and
the “A” and “C” rings are not both phenyl.in which the “A” or “C” ring is a pyridyl.

9. The method of claim 7 in which the PBI compound is selected from structures A, B, C and D of FIG. 10.

10. The method of claim 1 in which the NS5B polymerase is from an HCV genotype selected from the group consisting of HCV1a (H77), HCV1a(Chiron), HCV1b(J6), HCV1b(Con1), HCV2a, HCV2b, HCV3a, HCV4a, HCV5a and HCV6a.

11. A method of treating or preventing an HCV infection, comprising the step of administering to a subject in need thereof an amount of a PBI compound.

12. The method of claim 11 in which the PBI compound contacts, associates with and/or interacts with a region of the NS5B polymerase selected from:
an NS5B polymerase residue selected from positions 142, 148, 213, 316, 444, 445, 447, 451, 452, 465 and combinations thereof;
an NS5B polymerase residue positioned within alpha helix “O”, alpha helix “P”, alpha helix “R”, beta strand “17” and beta strand “18” as defined in FIG. 12;
an NS5B polymerase residue positioned within beta strand “17” and alpha helix “P”, “O” and/or “R” as defined in FIG. 12;
an NS5B polymerase residue positioned within beta strand “18” and alpha helix “P”, “O” and/or “R” as defined in FIG. 12; and
an NS5B polymerase residue positioned in a region defined by residues 440 to 470.

13. The method of claim 11 in which the PBI compound comprises the following structure: 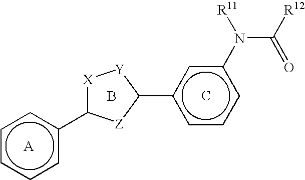
wherein:
the “A” ring is a substituted phenyl or pyridyl;
the “B” ring is saturated, unsaturated or aromatic and includes one or more heteroatoms at positions X, Y and Z which are selected from NH, N, O and S, with the proviso that X and Y are not both simultaneously O;
the “C” ring comprises a phenyl or pyridyl which may optionally include additional unillustrated substituents;
R¹¹ is hydrogen or alkyl; and
R¹² is mono- or di-halo methyl.

14. The method of claim 13 in which the PBI compound has one or more features selected from the group consisting of:
the “A” or “C” ring is a pyridyl;
the “A” and “C” rings are each phenyl; and
the “A” and “C” rings are not both phenyl.

15. The method of claim 14 in which the PBI compound is selected from structures A, B, C and D of FIG. 10.

16. The method of claim 11 in which the PBI compound is selected form the group consisting of an antibody or binding fragment thereof, a nucleic acid and an RNA.

17. The method of claim 11 in which the PBI compound competes for binding the NS5B polymerase with a second PBI compound comprising the structure: 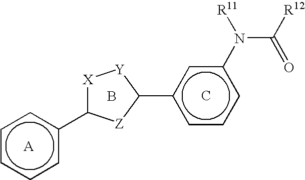
wherein:
the “A” ring is a substituted phenyl or pyridyl;
the “B” ring is saturated, unsaturated or aromatic and includes one or more heteroatoms at positions X, Y and Z which are selected from NH, N, O and S, with the proviso that X and Y are not both simultaneously O;
the “C” ring comprises a phenyl or pyridyl which may optionally include additional unillustrated substituents;
R¹¹ is hydrogen or alkyl; and
R¹² is mono- or di-halo methyl.

18. The method of claim 17 in which the second PBI compound has one or more features selected from the group consisting of:
the “A” or “C” ring is a pyridyl;
the “A” and “C” rings are each phenyl; and
the “A” and “C” rings are not both phenyl.in which the “A” or “C” ring is a pyridyl.

19. The method of claim 17 in which the PBI compound is selected from structures A, B, C and D of FIG. 10.

20. The method of claim 11 which is practiced therapeutically in a subject suffering from an HCV infection.

21. The method of claim 11 which is practiced prophylactically in a subject thought to be at risk of developing an HCV infection.

22. The method of claim 11 in which the HCV infection is caused by an HCV genotype selected from the group consisting of HCV1a (H77), HCV1a(Chiron), HCV1b(J6), HCV1b(Con1), HCV2a, HCV2b, HCV3a, HCV4a, HCV5a and HCV6a

23. A method of identifying a compound which inhibits HCV replication and/or proliferation, comprising:
contacting an HCV NS5B polymerase or a fragment thereof with a candidate compound; and
determining whether the candidate compound contacts, associates with and/or interacts with a region of the NS5B polymerase or fragment selected from the group consisting of:
an NS5B polymerase residue selected from positions 142, 148, 213, 316, 444, 445, 447, 451, 452, 465 and combinations thereof;
an NS5B polymerase residue positioned within alpha helix “O”, alpha helix “P”, alpha helix “R”, beta strand “17” and beta strand “18” as defined in FIG. 12;
an NS5B polymerase residue positioned within beta strand “17” and alpha helix “P”, “O” and/or “R” as defined in FIG. 12;
an NS5B polymerase residue positioned within beta strand “18” and alpha helix “P”, “O” and/or “R” as defined in FIG. 12; and
an NS5B polymerase residue positioned in a region defined by residues 440 to 470.

24. The method of claim 23 which is carried out in vitro.

25. The method of claim 24 in which the contacting is carried out in the presence of a PBI compound.

26. The method of claim 25 in which the PBI compound is selected from structures A, B, C and D of FIG. 10.

27. The method of claim 23 in which the NS5B polymerase is immobilized on a solid support.

28. The method of claim 23 in which the candidate compound or the PBI compound is labeled.

29. The method of claim 23 in which the candidate compound is immobilized on a solid support.

30. The method of claim 23 in which the NS5B polymerase is labeled.

31. The method of claim 30 in which the NS5B polymerase is ¹⁵N-labeled.

32. The method of claim 23 in which the determining step is carried out using NMR spectroscopy.

33. The method of claim 23 which is carried out in silico with structural coordinates comprising the pocket region of the NS5B polymerase.

34. A method of identifying a PBI compound, comprising the steps of:
superimposing a model of a candidate compound on a structural representation of the pocket region of an NS5B polymerase; and
assessing whether the candidate compound model fits spatially into the pocket region, wherein a spatial fit identifies the candidate compound as a PBI compound.

35. The method of claim 34 in which the pocket region of the NS5B polymerase is defined by the residues at positions 142, 148, 213, 316, 444, 445, 447, 451, 452 and 465.

36. The method of claim 34 in which the pocket region of the NS5B polymerase is defined by a region of the NS5B polymerase selected from the group consisting of:
beta strand “17” and alpha helix “O”, “P” and/or “R” as defined in FIG. 12;
strand “17,” beta strand “18”, alpha helix “O”, alpha helix “P” and alpha helix “R” as defined in FIG. 12;
beta strand “17”, beta strand “18” and alpha helix “O”, “P” and/or “R” as defined in FIG. 12;
beta strand “18”, alpha helix “O”, alpha helix “P” and alpha helix “R” as defined in FIG. 12; and
residues 440 to 470.

37. The method of any one of claim 34 in which the structural representation of the pocket region is derived from the structural coordinates of a full-length NS5B polymerase.

38. The method of any one of claims 34 which further includes the step of determining whether the identified PBI compound inhibits an activity of an NS5B polymerase in an activity assay.

39. A method of identifying a PBI compound, comprising the steps of computationally screening a three-dimensional representation of the pocket region of an NS5B polymerase with a candidate compound and determining whether the candidate compound binds the pocket region, wherein binding the pocket region identifies the candidate compound as a PBI compound.

40. The method of claim 39 in which the determining step comprises determining whether the candidate compound contacts, associates with and/or interacts with a region of the NS5B polymerase selected from:
an NS5B polymerase residue selected from positions 142, 148, 213, 316, 444, 445, 447, 451, 452, 465 and combinations thereof;
an NS5B polymerase residue positioned within alpha helix “O”, alpha helix “P”, alpha helix “R”, beta strand “17” and beta strand “18” as defined in FIG. 12a;
an NS5B polymerase residue positioned within beta strand “17” and alpha helix “P”, “O” and/or “R” as defined in FIG. 12;
an NS5B polymerase residue positioned within beta strand “18” and alpha helix “P”, “O” and/or “R” as defined in FIG. 12; and
an NS5B polymerase residue positioned in a region defined by residues 440 to 470.

41. The method of claim 39 in which the three-dimensional representation of the pocket region of the NS5B polymerase is derived from the atomic structure coordinates deposited at the Protein Data Bank under deposit nos. 1CSJ, 1C2P, 1QUV or provided in U.S. Pat. No. 6,434,489, or structure coordinates that have a root mean square deviation of the backbone atoms of the residues defining the pocket region that is less than 2 Å from any of the above coordinates.

42. The method of claim 40 in which a plurality of candidate compounds are screened.

43. A machine-readable medium embedded with atomic structure coordinates of a fragment of an NS5B polymerase, wherein said fragment comprises the residues defining the pocket region of the NS5B polymerase.

44. The machine-readable medium of claim 43 in which the fragment comprises residues 440 to 470.

45. The machine-readable medium of claim 43 in which the fragment is discontinuous and comprises residues 142, 148, 213, 316, 444, 445, 447, 451, 452 and 465.

46. The machine-readable medium of claim 43 in which the fragment is discontinuous and comprises a region of the NS5B polymerase selected from:
beta strand “17”, beta strand “18” and alpha helix “O”, “P” and/or “R” as defined in FIG. 12; and
beta strand “17”, beta strand “18”, alpha helix “O”, alpha helix “P” and alpha helix “R” as defined in FIG. 12.

47. A computer system for generating a three-dimensional representation of the pocket of an NS5B polymerase, comprising:
memory comprising atomic structure coordinates of a fragment of an NS5B polymerase, wherein said fragment comprises residues defining the pocket region;
a central-processing unit coupled to the memory; and
a display coupled to the central-processing unit for displaying the three-dimensional representation.

48. The computer system of claim 47 in which the fragment comprises residues 440 to 470.

49. The computer system of claim 47 in which the fragment is discontinuous and comprises residues 142, 148, 213, 316, 444, 445, 447, 451, 452 and 465.

50. The computer system of claim 47 in which the fragment is discontinuous and comprises a region of the NS5B polymerase selected from:
beta strand “17”, beta strand “18” and/or alpha helix “R” as defined in FIG. 12; and
beta strand “17”, beta strand “18”, alpha helix “O”, alpha helix “P” and alpha helix “R” as defined in FIG. 12.
